(19) United States
(12) Patent Application Publication
(10) Pub. No.: US 2003/0185854 A1
(43) Pub. Date: Oct. 2, 2003

(54) Use of recombinant hepatitis B core particles to develop vaccines against infectious pathogens and malignancies

(76) Inventors: Fidel Zavala, New York, NY (US); Ashley J. Birkett, Escondido, CA (US)

Correspondence Address: DARBY & DARBY P.C., P. O. BOX 5257, NEW YORK, NY, 10150-5257, US

(21) Appl. No.: 10/360,836

(22) Filed: Feb. 7, 2003

Related U.S. Application Data
(60) Provisional application No. 60/354,963, filed on Feb. 8, 2002.

(51) Int. Cl. A61K 39/12

(52) U.S. Cl. 424/199.1

(57) ABSTRACT

The present invention relates to methods and compositions for augmenting CD8+ T cell responses to an antigen in a mammal, comprising the use of recombinant hepatitis B core particles (rHEP) to present said antigen. The invention further relates to a method of boosting the rHEP particle-induced CD8+ T cell responses using secondary immunization with a recombinant vaccinia virus expressing the same antigen (rVAC). The methods and compositions of the present invention can be useful for prophylaxis and treatment of various infectious and neoplastic diseases.

### BACKGROUND OF THE INVENTION

### Vaccine Technology

The successful elimination of pathogens, neoplastic cells, or self-reactive immune mechanisms following prophylactic or therapeutic immunization depends to a large extent on the ability of the host's immune system to become activated in response to the immunization and mount an effective response, preferably with minimal injury to healthy tissue.

The rational design of vaccines initially involves identification of immunological correlates of protection—the immune effector mechanism(s) responsible for protection against disease—and the subsequent selection of an antigen that is able to elicit the desired adaptive response. Once this appropriate antigen has been identified, it is essential to deliver it effectively to the host's immune system.

New vaccines are presently under development and in testing for the control of various infectious and neoplastic diseases. In contrast to older vaccines which were typically based on live-attenuated or non-replicating inactivated pathogens, modern vaccines are composed of synthetic, recombinant, or highly purified subunit antigens (e.g., recombinant or synthetic polypeptides, nucleic acids, or recombinant bacterial or viral vectors capable of inducing antibodies as well as T cell responses [see, e.g., Liljeqvist and Sthal, J. Biotech, 73:1-33, 1999]). Subunit vaccines are designed to include only the antigens required for protective immunization and are believed to be safer than whole-inactivated or live-attenuated vaccines. However, the purity of the subunit antigens and the absence of the self-adjuvanting immunomodulatory components associated with attenuated or killed vaccines often result in weaker immunogenicity.

The immunogenicity of a relatively weak antigen can be enhanced by presenting it in conjunction with a carrier platform. As it has been demonstrated that, when exogenous antigens are particulate in nature, they are presented 1,000 to 10,000-fold more efficiently than soluble antigens in both the MHC class I and class II pathways (Harris et al., Immunology, 77:315-321, 1992; Griffiths et al., J Virol., 67:3191-3198, 1993; Schodel et al., Int. Rev. Immunol., 11:153-165, 1994; Schirmbeck et al., Eur. J. Immunol., 25:1063-1070, 1995; and Raychaudhuri and Rock, Nat. Biotechnol., 16:1025-1031, 1998), many currently used antigens are being incorporated into highly immunogenic particle forming protein carrier platforms such as hepatitis B core (HBcAg) (Clarke et al., Nature, 330: 381-384, 1987) or surface (HBsAg) (Delpeyroux et al., Science, 223: 472-475, 1986) antigen particles, the Ty element of yeast (Adams et al., Nature, 329: 68-70, 1988), or poliovirus virions (Burke et al., Nature, 332: 81-82, 1988).

Hepatitis B virus (HBV) core antigen (HBcAg) is a 21 kDa protein that self-assembles to form the subviral 30-32 nm nucleocapsid particles packaging the viral polymerase and pregenomic RNA during HBV replication. HBcAg also assembles to form particles when synthesized in the absence of other HBV gene products in a wide range of prokaryotic and eukaryotic recombinant expression systems. Two particle sizes are observed, a slightly smaller particle with a T=3 symmetry consisting of 180 subunits and a larger particle consisting of 240 subunits and displaying a T=4 symmetry (Crowther et al., Cell, 77: 943-950, 1994). In particles where the nucleic acid binding domain has been deleted, the latter is the predominant species. Apart from the ease of recombinant expression and self-assembly, HBcAg has several features that make it an attractive carrier moiety for foreign haptens (reviewed in Milich, Immunol. Today, 9: 380-386, 1998 and Schödel et al., Int. Rev. Immunol., 11: 153-164, 1994). Thus, recombinant expression systems for HBcAg have been developed and sites within HBcAg characterized that enhanced the immunogenicity of B cell epitopes inserted into recombinant hybrid HBcAg particles (Schödel et al., J. Virol. 66: 106-114, 1992). It has been demonstrated that HBcAg can directly activate B cells and elicits CD4+ T cell responses (see, e.g., Milich and McLachlan, Science 234:1398, 1986; Millich et al., J. Immunol. 139:1223, 1987; Milich et al., Nature 329:547, 1987; Milich, Proc. Natl. Acad. Sci. USA, 87:6599, 1990; Schodel et al., J. Virol., 66:106, 1992; Schodel et al., Int. Rev. Immunol., 11:153, 1994; Milich et al., Ann. N.Y. Acad. Sci., 754:187, 1995; also see, e.g., U.S. Pat. Nos.: 4,882,145; 4,882,145, and 5,143,726). In addition, it was shown that HBV-infected individuals mount a high titered anti-HBcAg antibody response indicative of an enhanced immunogenicity of HBcAg in man, at the same time its expression is apparently non-toxic. As the preexisting anti-HBcAg antibodies appear to have no negative effect on the immunogenicity of hybrid HBcAg-heterologous epitope particles, it is possible to employ these particles in a population that has been exposed to hepatitis B virus infection (Schodel et al., J. Exp. Med., 80:1037-1046, 1994).

For example, Schödel et al. (Behring Inst. Mitt., 98:114-119, 1997 and J. Virol. 66:106-114, 1992) inserted repeat B cell epitopes of the circumsporozoite antigens (CS) of Plasmodium berghei, P. yoelii and P. falciparum, into hybrid HBcAg-CS particles and immunized mice with such hybrid HBcAg-CS particles. These mice displayed high titered serum antibody responses to CS peptides as well as were protected against parasite challenge.

It has been demonstrated that heterologous amino acid sequences may be incorporated into HBcAg recombinant particles by creating N- or C-terminal fusions, or the internal fusions in the region corresponding to amino acids 75-85 (Pumpens et al., Intervirology, 38:63-74, 1995; Ulrich et al., Adv. Virus Res., 50: 141-182, 1998; Pumpens et al., Intervirology, 44:98-114, 2001) of HBcAg (according to recent structural data, this region is located at the tips of prominent surface spikes formed by the very stable dimer interfaces; see, e.g., Kratz et al., Proc. Natl. Acad. Sci. USA, 96:1915, 1999). C-teminal fusions allow for the longest sequences to be inserted (e.g., up to 720 amino acids, as disclosed in Pumpens et al., Intervirology, 38:63-74, 1995). It has been demonstrated that C-terminal amino acids 145-183 of HBcAg are not necessary for capsid assembly and do not affect the yield, size or morphology of HBcAg particles synthesized in E. coli, while truncation of more than 3 N-terminal amino acids of HBcAg results in complete disapperance of chimeric protein in E. coli cells (Pumpens et al., Intervirology, 38:63-74, 1995). However, in some cases, the internal insertions appear not only more immunogenic but also capable of reducing the level of anti-HBcAg antibodies produced, which may allow the recombinant particles to escape anti-carrier-antibody-mediated suppression of immune responses against the heterologous epitope (Schödel et al., J. Virol., 66:106-114, 1992; Schödel et al., Int. Rev. Immunol., 11:153-164, 1994). The internal site between amino acids 75 and 82 of HBcAg can accommodate heterologous sequences of up to 238 amino acids (Kratz et al., Proc. Natl. Acad. Sci. USA, 96:1915, 1999; Pumpens et al., Intervirology, 44:98-114, 2001)). However, whether or not insertion of a heterologous sequence is compatible with core particle assembly is not only dependent on the length of the sequence but also on the specific primary sequence context (Schödel et al., Intervirology, 39:104-110, 1996).

### CD8+ T Cell and Antibody Responses

CD8+ T cells represent one of the most important mechanisms of protective immunity against intracellular infectious agents such as viruses, bacteria and parasites (see, e.g., Nardin and Nussenzweig, Ann. Rev. Immunol. 11: 687-727, 1993; Harty et al., Ann. Rev. Immunol., 18:275-308, 2000; Hill et al., Nature 352: 595-600, 1991; Aidoo et al., The Lancet 345: 1003-1007, 1995; Wizel et al., J. Exp Med. 182: 1435-1445, 1995; Lalvani et al., Res. Immunol. 145: 461-468, 1994; Hel et al., J. Immunol., 167:7180-7191, 2001; Fujimura et al., Infect. Immun., 69:5477-5486, 2001; Kim et al., J. Immunol., 162:6855-6866, 1999). For example, in rodent malaria models, it has been well established that CD8+ T cells induced after immunization with attenuated or viable malaria sporozoites play an important role in protection against liver stages of this parasite (Romero et al., Nature, 341: 323-326, 1989; Rodrigues et al., Int. Immunol., 3: 579-585, 1991). It is also known that CD8+ T cells play an important role at preventing, controlling or even eliminating cells undergoing malignant transformation (Gorelik and Flavell, Nat. Med., 7:1118-1122, 2001; Hanson et al., Immunity, 13: 265, 2000).

There are two general types of CD8+ T cells: naive and memory cells. Naive and memory CD8+ T cells differ greatly with regards to their capacity to respond to antigenic stimulation. Activated memory T cells secrete cytokines and proliferate immediately after antigen recognition. In contrast, naive CD8+ T cells undergo a series of phenotypic changes before differentiating into effector cells (Veiga-Fernandes et al., Nat. Immunol., 1: 47-53, 2000; Iezzi et al., Immunity, 8: 89-95, 1998).

CD8+ T cells may function in more than one way. The best known function is the killing or lysis of target cells bearing peptide antigen in the context of an MHC class I molecule. Hence, these cells are often termed cytotoxic T lymphocytes (CTL). However, another function, perhaps of greater protective relevance in infections is the ability of CD8+ T cells to secrete cytokines (e.g., interferon gamma [IFN-γ]). Assays of lytic activity and of cytokine release are both of value in measuring a CD8+ T cell immune response.

Although many antigen delivery systems have been investigated (e.g., recombinant bacteria, viruses, naked DNA, RNA, as well as various coated particles and synthetic peptides), a significant in vivo expansion of primary CD8+ T cell responses proved difficult to achieve. Indeed, multiple research groups reported that, once the CD8+ T cell response is established, the number of antigen-specific T cells cannot be increased in spite of repeated administration of the same immunogen (Matloubian et al., J. Virol., 68: 8056-8063, 1994; Zimmerman et al., J. Exp. Med., 183:1367-1375, 1996; Murata et al., Cell. Immunol., 173: 96-107, 1996).

The realization that CD8+ T cells play such an important immune protective role has stimulated research aimed at the development of subunit vaccines specifically designed to induce CD8+ T cell-mediated immunity. Despite these efforts, currently, there is not a single vaccine that has been designed to efficiently induce CD8+ T cell immunity.

One of the main difficulties encountered in the development of this type of vaccines has been that most of the carriers that have been used, while capable of inducing a detectable response, induce responses of relatively small magnitude, not sufficient to confer in vivo protection (Zavala et al., Virology, 280:155-159, 2001). This is the case for DNA-based vaccine and for genetically engineered live viruses and bacteria. Another difficulty stems from the fact that, in general, vaccines that are capable of inducing CD8+ T cell responses appear to be inefficient at inducing antibody responses (Hel et al., J. Immunol., 167:7180-7191, 2001). This is an important issue since it is also well understood that effective vaccines should be capable of inducing both humoral (antibody-mediated) and cellular (T cell-mediated) immunity.

There is evidence that at least in some instances antibody and T cell responses can be improved by using an immunization strategy which combines different recombinant vectors expressing the same antigen or epitope, in particular, by using two different vectors administered sequentially as prime and boost (Li et al., Proc. Natl. Acad. Sci. USA, 90:5214-5218, 1993; Rodrigues et al., J. Immunol., 153:4636-4648, 1994; Murata et al., Cell. Immunol., 173:96-107, 1996; Schneider et al., Nat. Med., 4:397-402, 1998; Sedegah et al., Proc. Natl. Acad. Sci. USA, 95:7648-7654, 1998; Robinson et al., Nat. Med., 5:526-534, 1999; Ramshaw and Ramsay, Immunol. Today, 21:163-165, 2000; Hel et al., J. Immunol., 167:7180-7191, 2001; Pancholi et al., Hepatology, 33:448-454, 2001 and J. Infect. Dis., 182:18-27, 2000).

Evidence that a heterologous prime-boost immunization regimen might affect CD8+ T cell responses was provided by Li et al. (Proc. Natl. Acad. Sci. USA, 90:5214-5218, 1993) who showed that priming mice with recombinant influenza virus (reFlu) expressing a CD8+ T cell epitope of the Plasmodium yoelii CS protein followed by a booster with recombinant vaccinia viruses (reVV) expressing the same epitope enhanced greatly the specific CD8+ T cell response. Mice immunized according to this protocol displayed not only a strong secondary CS-specific CD8+ T cell response but also a considerable degree of protection against malaria infection (see also Rodrigues et al, J. Immunol., 153:4636-4648, 1994; Murata et al., Cell. Immunol., 173:96-107, 1996). Similar results were obtained in mice immunized with irradiated P. yoelii or P. falciparum sporozoites, in which the CS-specific CD8+ T cell responses could be increased 10- to 20-fold after booster with a reVV expressing the CS epitope (Miyahira et al., Proc. Natl. Acad. Sci. USA, 95:3954-3959, 1998). The prime/boost regimen using a non-replicating poxvirus to boost the immune response elicited by a different vector has also been successfully applied in macaques. Immunization of macaques (Macaca nemestrina) by priming with DNA and boosting with a recombinant fowlpox (reFPV), both encoding HIV-1 env, gag, and pol antigens, was found to generate an enhancement of HIV-1-specific CTL and T helper (Th) responses and protection against a non-pathogenic HIV-1 challenge (Kent et al., J. Virol., 72:10180-10188, 1998; see also Hanke et al., J. Virol., 73:7524-7532, 1999 and Robinson et al., Nat. Med., 5:526-534, 1999). Neither of these two vectors was by itself able to generate a consistent CTL response.

In recent studies by the present inventors and co-workers using P. yoelii malaria model, it was shown that priming with recombinant virus-like particles (VLPs) derived from a yeast retrotransposon (TyVLPs), carrying the P. yoelii CS CTL epitope, followed by boosting with a reVV expressing the CS protein, induced strong secondary CD8+ T cell responses that protected 62% of mice against sporozoite challenge (Oliveira-Ferreira et al., Vaccine, 18:1863-1869, 2000).

The enhancement provided by recombinant vaccinia virus is not restricted to malaria antigens since enhancement also occurred in mice primed with influenza virus and boosted with a reVV expressing the influenza nucleoprotein (NP). The combined immunization also resulted in a greatly enhanced secondary anti-NP-specific CD8+ T cell response (Murata et al., Cell. Immunol., 173:96-107, 1996; see also Gonzalo et al., Vaccine, 17:887-892, 1999; Zavala et al., Virology, 280:155-159, 2001).

However, there are two limitations to these findings in terms of their potential usefulness: the immunogenicity induced was only sufficient to achieve partial protection against malaria and was dependent on a highly immunogenic priming immunization with a replicating recombinant influenza virus, which causes numerous side-effects upon administration making it not suitable for general human use in vaccines.

In studies in which the immunogenicity of different viral vectors expressing the CD8+ T cell epitope of the P. yoelii CS protein was compared, it was found that each of these viral vectors, i.e., Sindbis, adeno, influenza and vaccinia viruses, induced a considerable CS-specific CD8+ primary response (Murata et al., Cell. Immunol., 173:96-107, 1996; Rodrigues et al., J. Immunol., 158:1268-1274, 1997; Tsuji et al., J. Virol., 72:6907-6910, 1998). However, the in vivo expansion of an established CD8+ T cell response was difficult to achieve. The fact that other recombinant viruses (Murata et al., Cell. Immunol., 173:96-107, 1996), DNA (Schneider et al., Nat. Med., 4:397-402, 1998), synthetic peptides (Miyahira et al., Proc. Natl. Acad. Sci. USA, 95:3954-3959, 1998), and virus-like particles (Oliveira-Ferreira et al., Vaccine, 18:1863-1869, 2000) are incapable or rather inefficient at inducing large secondary CD8+ T cell responses suggests the existence of severe constraints for the in vivo expansion of memory T cells. These studies also indicated that, compared to other vectors, recombinant vaccinia viruses (in particular, non-replicating or replication-impaired viruses) are particularly efficient in boosting the memory CD8+ T cells responses.

Accordingly, there is a great need in the art to develop a vaccine and an efficient boosting regimen capable of inducing both antibodies and T cell responses, in particular, CD8+ T cell responses, wherein the T cell responses are designed to invoke a specific response that is engineered to invoke a Th1 or Th2 response, depending on the nature of pathogen and the invasiveness of the infection.

The present invention addresses these and other needs by providing for the first time methods and compositions for augmenting CD8+ T cell responses to an antigen in a mammal, comprising the use of recombinant hepatitis B core particles (rHEP) to present a CD8+ T cell epitope of said antigen. The present invention further provides an efficient method of boosting the rHEP particle-induced CD8+ T cell responses using secondary immunization with a non-replicating or replication-impaired recombinant vaccinia virus expressing the same CD8+ T cell epitope (rVAC).

### SUMMARY OF THE INVENTION

The primary object of the present invention is to provide a method for generating an immune response against a non-hepatitis B, preferably non-hepadnaviral, antigen in a mammal, which method comprises administering to the mammal at least one dose of a priming component comprising a recombinant hepatitis B core particle (rHEP) which is a carrier for one or more non-hepatitis B, preferably non-hepadnaviral, CD8+ T cell epitopes of the antigen, wherein administering the priming component induces an antigen-specific CD8+ T cell immune response.

According to the present invention, the use of hepatitis B core particle carrier platform results in an enhancement of the immunity induced by the antigen and is attributed at least in part to the enhancement of antigen-specific CD8+ T cell responses.

In a preferred embodiment, the method of the invention comprises two steps, wherein the administration of the priming component is followed by administering at least one dose of a boosting component comprising a carrier for one or more CD8+ T cell epitopes of the antigen, including at least one CD8+ T cell epitope which is the same as the CD8+ T cell epitope of the priming component. Preferably, the boosting component to be used according to the invention is a non-replicating or replication-impaired recombinant poxvirus vector, most preferably vaccinia strain modified virus Ankara (MVA), or a strain derived therefrom, or NYVAC vaccinia strain.

As disclosed herein, either priming or boosting component or both can additionally contain one or more non-CD8+ epitopes of the antigen, such as, for example, a CD4+ T cell epitope or a B cell epitope.

As disclosed herein, the priming and boosting components are administered sequentially. Preferably, the boosting component is administered from two weeks to four months after the priming component.

In a specific embodiment, the invention provides a method for conferring immunity against the sporozoite stage of malaria to a susceptible mammalian host comprising administering to said host (i) a priming component comprising a recombinant hepatitis B core particle (rHEP) which is a carrier for one or more non-hepatitis B CD8+ T cell epitopes of at least one plasmodial sporozoite antigen in a first amount, and (ii) a boosting component comprising a carrier for one or more CD8+ T cell epitopes of the antigen, including at least one CD8+ T cell epitope which is the same as the CD8+ T cell epitope of the priming component in a second amount; said first and second amounts being effective in combination to enhance the immune response mounted against said plasmodial sporozoite antigen by the host. Preferably, the boosting component is a non-replicating or replication-impaired recombinant poxvirus vector. Also preferably, the malaria-specific CD8+ T cell epitope has an amino acid sequence selected from the group consisting of SYVPSAEQI (SEQ ID NO: 1), SYIPSAEKI (SEQ ID NO: 2), YNRNIVNRLLGDALNGKPEEK (SEQ ID NO: 3), EYLNKIQNSLSTEWSPCSVT (SEQ ID NO: 4), KPKDELDYENDIEKKICKMEKCS (SEQ ID NO: 5), MNHLGNVKYLVIVFL (SEQ ID NO: 6), EVDLYLLMDCSGSIR (SEQ ID NO: 7), LLSTNLPYGKTNLTD (SEQ ID NO: 8), LPYGKTNLTDALLQV (SEQ ID NO: 9), TNLTDALLQVRKHLN (SEQ ID NO: 10), ALLQVRKHLNDRINR (SEQ ID NO: 11), ENVKNVIGPFMKAVC (SEQ ID NO: 12), CEEERCLPKREPLDV (SEQ ID NO: 13), CLPKREPLDVPDEPE (SEQ ID NO: 14), ALLACAGLAYKFVVP (SEQ ID NO: 15), APFDETLGEEDKDLD (SEQ ID NO: 16), TLGEEDKDLDEPEQF (SEQ ID NO: 17), ASKNKEKAL (SEQ ID NO: 18), KNKEKALII (SEQ ID NO: 19), FLIFFDLFLV (SEQ ID NO: 20), VLAGLLGNV (SEQ ID NO: 21), GLIMVLSFL (SEQ ID NO: 22), KILSVFFLA (SEQ ID NO: 23), GLLGNVSTV (SEQ ID NO: 24), VLLGGVGLVL (SEQ ID NO: 25), ILSVSSFLFV (SEQ ID NO: 26), QTNFKSLLR (SEQ ID NO: 27), LACAGLAYK (SEQ ID NO: 28), VTCGNGIQVR (SEQ ID NO: 29), ALFFIIFNK (SEQ ID NO: 30), LLACAGLAYK (SEQ ID NO: 31), GVSENIFLK (SEQ ID NO: 32), HVLSHNSYEK (SEQ ID NO: 33), FILVNLLIFH (SEQ ID NO: 34), MPLETQLAI (SEQ ID NO: 35), TPYAGEPAPF (SEQ ID NO: 36), DLLEEGNTL (SEQ ID NO: 37), KLEELHENV (SEQ ID NO: 38), VLDKVEETV (SEQ ID NO: 39), GLLNKLENI (SEQ ID NO: 40), MEKLKELEK (SEQ ID NO: 41), EPKDEIVEV (SEQ ID NO: 42), and ATSVLAGL (SEQ ID NO: 43).

In another specific embodiment, the invention provides a method for conferring immunity against the influenza virus to a susceptible mammalian host comprising administering to said host (i) a priming component comprising a recombinant hepatitis B core particle (rHEP) which is a carrier for one or more non-hepatitis B CD8+ T cell epitopes of at least one influenza virus-specific antigen in a first amount, and (ii) a boosting component comprising a carrier for one or more CD8+ T cell epitopes of the antigen, including at least one CD8+ T cell epitope which is the same as the CD8+ T cell epitope of the priming component in a second amount; said first and second amounts being effective in combination to enhance the immune response mounted against said influenza virus-specific antigen by the host. Preferably, the boosting component is a non-replicating or replication-impaired recombinant poxvirus vector. Also preferably, the influenza virus-specific CD8+ T cell epitope has an amino acid sequence of the influenza A virus nucleoprotein (NP), most preferably, selected from the group consisting of TYQRTRALV (SEQ ID NO: 44) and SDYEGRLI (SEQ ID NO: 45).

In conjunction with the methods of the present invention, further provided are pharmaceutical and vaccine compositions comprising an immunogenically effective amount of a priming component comprising a recombinant hepatitis B core particle (rHEP) which is a carrier for one or more non-hepatitis B, preferably non-hepadnaviral, CD8+ T cell epitopes of a non-hepatitis B, preferably non-hepadnaviral, antigen and, optionally, further comprising a pharmaceutically acceptable adjuvant or excipient. As specified above, the priming component can additionally contain one or more non-CD8+ epitopes of the antigen, such as, for example, a CD4+ T cell epitope or a B cell epitope.

Also provided herein is a method for augmenting the immunity induced by an antigen in a mammal comprising administering to said mammal the pharmaceutical composition of the invention and, optionally, further comprising administering an immunogenically effective amount of a boosting component comprising a carrier for one or more CD8+ T cell epitopes of the antigen, including at least one CD8+ T cell epitope which is the same as the CD8+ T cell epitope of the priming component.

As disclosed herein, the antigen to be used according to the invention is selected from the group consisting of viral antigens, bacterial antigens, protozoan antigens, cancer antigens, and fungal antigens. In one of the embodiments, the antigen is a malaria-specific antigen, which preferably comprises a CD8+ T cell epitope of the plasmodial circumsporozoite (CS) protein. In another embodiment, the antigen is an influenza virus-specific antigen, which preferably comprises a CD8+ T cell epitope of the influenza virus nucleoprotein (NP).

In a further embodiment, the invention provides a prophylactic and/or therapeutic method for treating a disease in a mammal comprising administering to said mammal at least one dose of the priming component comprising a recombinant hepatitis B core particle (rHEP) which is a carrier for one or more non-hepatitis B, preferably non-hepadnaviral, CD8+ T cell epitopes of an antigen. Preferably, administering of the priming component is followed by administering at least one dose of a boosting component comprising a carrier for one or more CD8+ T cell epitopes of the antigen, including at least one CD8+ T cell epitope which is the same as the CD8+ T cell epitope of the priming component. As specified herein, this method can be useful for preventing and/or treating various infectious or neoplastic diseases. In a specific embodiment, the method of the invention is employed to treat an infection selected from the group consisting of viral infection, bacterial infection, parasitic infection, and fungal infection.

In a related embodiment, the present invention provides a kit for conferring immunity against a non-hepatitis B, preferably non-hepadnaviral, antigen in a mammal comprising (i) a pharmaceutical composition comprising a priming component comprising a recombinant hepatitis B core particle (rHEP) which is a carrier for one or more non-hepatitis B, preferably non-hepadnaviral, CD8+ T cell epitopes of the antigen in a first amount, and (ii) a pharmaceutical composition comprising a boosting component comprising a carrier for one or more CD8+ T cell epitopes of the antigen, including at least one CD8+ T cell epitope which is the same as the CD8+ T cell epitope of the priming component in a second amount; said kit comprising the priming component in a first container, and the boosting component in a second container, and, optionally, instructions for administration of the components; and wherein optionally the containers are in a package.

### BRIEF DESCRIPTION OF THE DRAWINGS

FIG. 1 shows the frequency of SYVPSAEQI-specific CD8+ T cell responses determined by ELISPOT assay 10 days after boosting in mice (1) immunized with a recombinant hepatitis B core antigen (rHEP) containing the SYVPSAEQI epitope (SEQ ID NO: 1) of the P. yoelii circumsporozoite protein and boosted with a recombinant vaccinia virus expressing the same epitope (rVAC); (2) immunized with rVAC and boosted with rHEP; (3) immunized with rHEP and boosted with rHEP; (4) not immunized and boosted with rVAC.

FIG. 2 shows the frequency of SYVPSAEQI-specific CD8+ T cell responses determined by ELISPOT assay 8 days after boosting in mice (1) immunized with rHEP and boosted with irradiated sporozoites; (2) immunized with irradiated sporozoites and boosted with rHEP; (3) not immunized and boosted with irradiated sporozoites; (4) not immunized and boosted with rHEP.

FIG. 3 shows the frequency of TYQRTRALV-specific CD8+ T cell responses determined by ELISPOT assay 10 days after boosting in mice (1) immunized with a recombinant hepatitis B core antigen particles (CorVax-1690) containing the epitope TYQRTRALV (SEQ ID NO: 44) of the nucleoprotein of influenza A virus and not boosted; (2) immunized with CorVax-1690 and boosted with a recombinant vaccinia virus expressing the entire nucleoprotein from influenza A virus (FluVac); (3) immunized with CorVax-1690 and boosted with a recombinant vaccinia virus without an insert (wtVac); (4) immunized with FluVac and not boosted.

### DETAILED DESCRIPTION OF THE INVENTION

The present invention provides for the first time methods and compositions for augmenting CD8+ T cell responses to an antigen in a mammal, comprising the use of recombinant hepatitis B core particles (rHEP) to present a CD8+ T cell epitope of said antigen. As summarized in the Background section, while it is widely accepted that hepatitis B core particles are efficient carrier platforms for inducing antibody responses against heterologous B cell epitopes and raising CD4+ T cell responses, their capacity to efficiently induce CD8+ T cell responses has not been recognized or demonstrated. In fact, a number of researchers suggested that HBcAg is an unlikely carrier platform to be used for this purpose. For example, Street et al. (Arch. Virol., 144:1323-1343, 1999) have recently published an observation that recombinant hepatitis B core particles containing CTL epitopes of the E7 protein of human papilloma virus failed to prime E7-directed CTL responses when used to immunize mice for antigen processing through either the endogenous pathway via a Salmonella typhimurium vector, or through the exogenous pathway by parenteral immunization with a recombinant core. Similarly, Kuhober et al. (J. Immunol., 156:3687-3695, 1996) reported that immunization with exogenous HBcAg particles efficiently primed serum antibody responses but did not elicit CTL responses in H-2b mice.

In one aspect the present invention provides a method for generating an immune response against at least one target heterologous (i.e., non-hepatitis B, preferably non-hepadnaviral) antigen in a mammal, which method comprises administering at least one dose of a priming component comprising a recombinant hepatitis B core particle (rHEP) which is a carrier for one or more non-hepatitis B, preferably non-hepadnaviral, CD8+ T cell epitopes of the antigen, wherein administering the priming component induces an antigen-specific CD8+ T cell immune response; said administration optionally followed by at least one dose of a boosting component comprising a carrier for one or more CD8+ T cell epitopes of the target antigen, including at least one CD8+ T cell epitope which is the same as a CD8+ T cell epitope of the priming component. In one embodiment of the invention, the priming and, optionally, boosting components can additionally contain non-CD8+ epitopes of the target antigen, such as, e.g., CD4+ T cell epitopes, B cell epitopes, etc.

As disclosed herein, the priming and boosting components are administered sequentially. Preferably, the boosting component is administered from two weeks to four months after the priming component.

In conjunction with the methods of the present invention, also provided are pharmaceutical and vaccine compositions comprising an immunogenically effective amount of an rHEP antigenic particle as well as, optionally, an adjuvant or excipient (preferably, all pharmaceutically acceptable). Said antigen and adjuvant can be either formulated as a single composition or as two separate compositions, which can be administered conjointly, i.e., simultaneously or sequentially.

The present invention further provides pharmaceutical and vaccine compositions comprising a boosting component comprising a carrier for one or more CD8+ T cell epitopes of the antigen, including at least one CD8+ T cell epitope which is the same as the CD8+ T cell epitope of the priming component. Optionally, the boosting component can also contain an adjuvant or excipient (preferably, all pharmaceutically acceptable). In a preferred embodiment, the boosting component is a non-replicating or replication-impaired recombinant poxvirus vector. Particularly preferred is a vaccinia strain modified virus Ankara (MVA), which has a good safety record and does not replicate in most cell types and normal human tissues, or a strain derived therefrom, or NYVAC vaccinia strain. Other vaccinia vectors which are useful in the compositions of the present invention include but are not limited to avipox vectors such as fowlpox or canarypox vectors (e.g., ALVAC commercially available as Kanapox) or strains derived therefrom (e.g., as described by Pancholi et al., Hepatology 33:448-454, 2001 and J. Infect. Dis., 182:18-27, 2000). Alternatively, the boosting component can be a recombinant virus-like particle (VLPs) derived from, e.g., yeast retrotransposon (TyVLPs), a non-replicating adenovirus such as E1 deletion mutant, a viral vector based on herpes virus or Venezuelan equine encephalitis virus (VEE), a whole-inactivated or live-attenuated microbial agent (e.g., irradiated sporozoites as disclosed in Example 1, infra, or bacterial vectors based on recombinant BCG or recombinant Salmonella as described by Darji et al. [Cell, 91:765-775, 1997]).

### Definitions

As used herein, the term “immunogenic” means that an agent is capable of eliciting a humoral or cellular immune response, and preferably both, when administered to an animal having an immune system.

The term “antigen” refers to any agent (e.g., protein, peptide, polysaccharide, glycoprotein, glycolipid, nucleic acid, or combination thereof) that, when introduced into a host, animal or human, having an immune system (directly or upon expression as in, e.g., DNA vaccines), is recognized by the immune system of the host and is capable of specific immune reaction. As defined herein, the antigen-specific immune response can be humoral or cell-mediated, or both. An agent is termed “antigenic” when it is capable of specifically interacting with an antigen recognition molecule of the immune system, such as an immunoglobulin (antibody) or T cell antigen receptor (TCR). A molecule that is antigenic need not be itself immunogenic, i.e., capable of eliciting an immune response without an adjuvant or excipient.

The term “epitope” or “antigenic determinant” refers to any portion of an antigen recognized either by B cells, or T cells, or both. Preferably, interaction of an epitope with an antigen recognition site of an immunoglobulin or TCR involves antigen-specific immune recognition.

T cells recognize proteins only when they have been cleaved into smaller peptides and are presented in a complex called the “major histocompatability complex (MHC)” located on another cell's surface. There are two classes of MHC complexes-class I and class II, and each class is made up of many different alleles. Class I MHC complexes are found on virtually every cell and present peptides from proteins produced inside the cell. Thus, class I MHC complexes are useful for killing cells infected by viruses or cells which have become cancerous. T cells which have a protein called CD8 on their surface, i.e., CD8+ T cells, bind specifically to the MHC class I/peptide complexes via the TCR. This leads to cytolytic effector activities. Class II MHC complexes are found only on antigen-presenting cells (APC) and are used to present peptides from circulating pathogens which have been endocytosed by APCs. T cells which have a protein called CD4 on their surface, i.e., CD4+ T cells, bind to the MHC class I/peptide complexes via TCR. This leads to the synthesis of specific cytokines which stimulate an immune response. To be effectively recognized by the immune system via MHC class I presentation, an antigenic polypeptide has to contain an epitope of at least about 8 to 10 amino acids, while to be effectively recognized by the immune system via MHC class II presentation, an antigenic polypeptide has to contain an epitope of at least about 13 to 25 amino acids. See, e.g., Fundamental Immunology, 3^{rd} Edition, W. E. Paul ed., 1999, Lippincott-Raven Publ.

The term “species-specific antigen” refers to an antigen that is only present in or derived from a particular species. Thus, the term “malaria-derived” or “malaria-specific” antigen refers to a natural (e.g., irradiated sporozoites) or synthetic (e.g., chemically or recombinantly synthesized polypeptide) antigen comprising at least one epitope (B cell and/or T cell) derived from any one of the proteins constituting plasmodium (said plasmodium being without limitation P. falciparum, P. vivax, P. malariae, P. ovale, P. reichenowi, P. knowlesi, P. cynomolgi, P. brasilianum, P. yoelii, P. berghei, or P. chabaudi) and comprising at least 8 amino acid residues. A preferred plasmodial protein for antigen generation is circumsporozoite (CS) protein, however, other proteins can be also used, e.g., the Erythrocyte Secreted Protein-1 or -2 (PvESP-1 or PvESP-2), Thrombospondin Related Adhesion (Anonymous) protein (TRAP), also called Sporozoite Surface Protein 2 (SSP2), liver stage antigen 1 (LSA-1), liver stage antigen 3 (LSA-3), exported protein 1 (EXP 1), hsp70, SALSA, sporozoite threonine- and asparagine-rich protein (STARP), Hep17, MSA, RAP-1, RAP-2, etc. The antigens and epitopes of the present invention are termed “non-hepatitis B”, meaning that they are not present in or derived from hepatitis B virus. Preferably, the antigens of the invention are termed “non-hepadnaviral”, meaning that they are not present in or derived from hepadnaviral species.

The term “vaccine” refers to a composition (e.g., protein or vector) that can be used to elicit immunity in a recipient. It should be noted that to be effective, a vaccine of the invention can elicit immunity in a portion of the immunized population, as some individuals may fail to mount a robust or protective immune response, or, in some cases, any immune response. This inability may stem from the individual's genetic background or because of an immunodeficiency condition (either acquired or congenital) or immunosuppression (e.g., due to treatment with chemotherapy or use of immunosuppressive drugs, e.g., to prevent organ rejection or suppress an autoimmune condition). Vaccine efficacy can be established in animal models.

As disclosed herein, vaccine compositions of the invention comprise a “priming component”, i.e., the component capable of inducing an initial immune response. The priming component of the compositions of the instant invention contains a “recombinant hepatitis B core particle (rHEP)”, which is a fusion protein or a conjugate comprising a portion of hepatitis B core antigen (HBcAg) sufficient for hepatitis B core particle formation and one or more non-hepatitis B, preferably non-hepadnaviral, CD8+ T cell epitopes of the heterologous (i.e., non-hepatitis B, preferably non-hepadnaviral) antigen(s) of the invention. As disclosed herein, the priming component can additionally contain one or more non-CD8+ epitopes of the antigen(s), such as, for example, CD4+ T cell epitopes or B cell epitopes.

Vaccine compositions of the invention can further comprise a “boosting component”, i.e., the component capable of enhancing an initial immune response induced by the priming component. The boosting component of the compositions of the instant invention comprises a carrier for one or more CD8+ T cell epitopes of the antigen, including at least one CD8+ T cell epitope which is the same as the CD8+ T cell epitope of the priming component. Preferably, the boosting component is a non-replicating or replication-impaired recombinant poxvirus vector.

The term “DNA vaccine” is an informal term of art, and is used herein to refer to a vaccine delivered by means of a recombinant vector. An alternative, and more descriptive term used herein is “vector vaccine” (since some potential vectors, such as retroviruses and lentiviruses are RNA viruses, and since in some instances non-viral RNA instead of DNA is delivered to cells through the vector). Generally, the vector is administered in vivo, but ex vivo transduction of appropriate antigen presenting cells, such as dendritic cells (DC), with administration of the transduced cells in vivo, is also contemplated.

The term “treat” is used herein to mean to relieve or alleviate at least one symptom of a disease in a subject. Within the meaning of the present invention, the term “treat” may also mean to prolong the prepatency, i.e., the period between infection and clinical manifestation of a disease. The term “protect” is used herein to mean prevent or treat, or both, as appropriate, development or continuance of a disease in a subject. Within the meaning of the present invention, the disease is selected from the group consisting of infection (e.g., viral, bacterial, parasitic, or fungal) and malignancy (e.g., solid or blood tumors such as sarcomas, carcinomas, gliomas, blastomas, pancreatic cancer, breast cancer, ovarian cancer, prostate cancer, lymphoma, leukemia, melanoma, etc.). For example, as disclosed herein, a prophylactic administration of an anti-malarial vaccine comprising an immunogenically effective amount of a priming component comprising a recombinant hepatitis B core particle (rHEP) which is a carrier for one or more non-hepatitis B CD8+ T cell epitopes of a malaria-specific antigen can protect a recipient subject at risk of developing malaria. Similarly, according to the present invention, a therapeutic administration of the pharmaceutical composition comprising an immunogenically effective amount of a priming component comprising rHEP which is a carrier for one or more non-hepatitis B CD8+ T cell epitopes of a tumor-specific antigen can enhance an anti-tumor immune response leading to slow-down in tumor growth and metastasis or even tumor regression.

The term “protective immunity” refers to an immune response in a host animal (either active/acquired or passive/innate, or both) which leads to inactivation and/or reduction in the load of said antigen and to generation of long-lasting immunity (that is acquired, e.g., through production of antibodies), which prevents or delays the development of a disease upon repeated exposure to the same or a related antigen. A “protective immune response” involves humoral (antibody) immunity or cellular immunity, or both, effective to, e.g., eliminate or reduce the load of a pathogen or infected cell (or produce any other measurable alleviation of the infection), or to reduce a tumor burden in an immunized (vaccinated) subject. Within the meaning of the present invention, protective immunity may be partial.

Immune systems are classified into two general systems, the “innate” or “natural” immune system and the “acquired” or “adaptive” immune system. It is thought that the innate immune system initially keeps the infection under control, allowing time for the adaptive immune system to develop an appropriate response. Recent studies have suggested that the various components of the innate immune system trigger and augment the components of the adaptive immune system, including antigen-specific B and T lymphocytes (Fearon and Locksley, supra; Kos, 1998, Immunol. Res., 17:303; Romagnani, 1992, Immunol. Today, 13:379; Banchereau and Steinman, 1988, Nature, 392:245).

The term “innate immunity” or “natural immunity” refers to innate immune responses that are not affected by prior contact with the antigen. The main protective mechanisms of the innate immunity are the skin (protects against attachment of potential environmental invaders), mucous (traps bacteria and other foreign material), gastric acid (destroys swallowed invaders), antimicrobial substances such as interferon (IFN) (inhibits viral replication) and complement proteins (promotes bacterial destruction), fever (intensifies action of interferons, inhibits microbial growth, and enhances tissue repair), natural killer (NK) cells(destroy microbes and certain tumor cells, and attack certain virus infected cells), and the inflammatory response (mobilizes leukocytes such as macrophages and dendritic cells to phagocytose invaders). Some cells of the innate immune system, including macrophages and dendritic cells (DC), function as part of the adaptive immune system as well by taking up foreign antigens through pattern recognition receptors, combining peptide fragments of these antigens with MHC class I and class II molecules, and stimulating naive CD8+ and CD4+ T cells respectively (Banchereau and Steinman, supra; Holmskov et al., 1994, Immunol. Today, 15: 67; Ulevitch and Tobias, 1995, Annu. Rev. Immunol., 13: 437). Professional antigen-presenting cells (APC) communicate with these T cells leading to the differentiation of naive CD4+ T cells into T-helper 1 (Th1) or T-helper 2 (Th2) lymphocytes that mediate cellular and humoral immunity, respectively (Trinchieri, 1995, Annu. Rev. Immunol., 13: 251; Howard and O'Garra, 1992, Immunol. Today, 13: 198; Abbas et al., 1996, Nature, 383: 787; Okamura et al., 1998, Adv. Immunol., 70: 281; Mosmann and Sad, 1996, Immunol. Today, 17: 138; O'Garra, 1998, Immunity, 8: 275).

The term “acquired immunity” or “adaptive immunity” is used herein to mean active or passive, humoral or cellular immunity that is established during the life of an animal, is specific for the inducing antigen, and is marked by an enhanced response on repeated encounters with said antigen. A key feature of the T lymphocytes of the adaptive immune system is their ability to detect minute concentrations of pathogen-derived peptides presented by MHC molecules on the cell surface.

As used herein, the term “augment the immune response” means enhancing or extending the duration of the immune response, or both.

The phrase “enhance immune response” within the meaning of the present invention refers to the property or process of increasing the scale and/or efficiency of immunoreactivity to a given antigen, said immunoreactivity being either humoral or cellular immunity, or both. An immune response is believed to be enhanced, if any measurable parameter of antigen-specific immunoreactivity (e.g., antibody titer, T cell production) is increased at least two-fold, preferably ten-fold, most preferably thirty-fold.

The term “therapeutically effective” applied to dose or amount refers to that quantity of a compound or pharmaceutical composition or vaccine that is sufficient to result in a desired activity upon administration to a mammal in need thereof. As used herein with respect to antigen-containing compositions or vaccines, the term “therapeutically effective amount/dose” is used interchangeably with the term “immunogenically effective amount/dose” and refers to the amount/dose of a compound (e.g., an antigen presented as part of rHEP) or pharmaceutical composition or vaccine that is sufficient to produce an effective immune response upon administration to a mammal.

The phrase “pharmaceutically acceptable”, as used in connection with compositions of the invention, refers to molecular entities and other ingredients of such compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “adjuvant” and “immunoadjuvant” are used interchangeably in the present invention and refer to a compound or mixture that may be non-immunogenic when administered to a host alone, but that augments the host's immune response to another antigen when administered conjointly with that antigen.

The adjuvant of the invention can be administered as part of a pharmaceutical or vaccine composition comprising an antigen or as a separate formulation, which is administered conjointly with a second composition containing an antigen. The adjuvants of the invention include, but are not limited to, oil-emulsion and emulsifier-based adjuvants such as complete Freund's adjuvant, incomplete Freund's adjuvant, MF59, or SAF; mineral gels such as aluminum hydroxide (alum), aluminum phosphate or calcium phosphate; microbially-derived adjuvants such as cholera toxin (CT), pertussis toxin, Escherichia coli heat-labile toxin (LT), mutant toxins (e.g., LTK63 or LTR72), Bacille Calmette-Guerin (BCG), Corynebacterium parvum, DNA CpG motifs, muramyl dipeptide, or monophosphoryl lipid A; particulate adjuvants such as immunostimulatory complexes (ISCOMs), liposomes, biodegradable microspheres, or saponins (e.g., QS-21); cytokines such as IFN-γ, IL-2, IL-12 or GM-CSF; synthetic adjuvants such as nonionic block copolymers, muramyl peptide analogues (e.g., N-acetyl-muramyl-L-threonyl-D-isoglutamine [thr-MDP], N-acetyl-nor-muramyl-L-alanyl-D-isoglutamine, N-acetylmuramyl-L-alanyl-D-isoglutaminyl-L-alanine-2-[1′-2′-dipalmitoyl-sn-glycero-3-hydroxyphosphoryloxy]-ethylamine), polyphosphazenes, or synthetic polynucleotides, and surface active substances such as lysolecithin, pluronic polyols, polyanions, peptides, hydrocarbon emulsions, or keyhole limpet hemocyanins (KLH). Preferably, these adjuvants are pharmaceutically acceptable for use in humans.

Within the meaning of the present invention, the term “conjoint administration” is used to refer to administration of an immune adjuvant and an antigen simultaneously in one composition, or simultaneously in different compositions, or sequentially.

The term “excipient” applied to pharmaceutical or vaccine compositions of the invention refers to a diluent or vehicle with which an antigen-containing compound and/or an adjuvant is administered. Such pharmaceutical excipients can be sterile liquids, such as water and oils, including those of petroleum, animal, vegetable or synthetic origin, such as peanut oil, soybean oil, mineral oil, sesame oil and the like. Water or aqueous solution, saline solutions, and aqueous dextrose and glycerol solutions are preferably employed as excipients, particularly for injectable solutions. Suitable pharmaceutical excipients are described in “Remington's Pharmaceutical Sciences” by E. W. Martin, 18^{th} Edition.

The term “native antibodies” or “immunoglobulins” refers to usually heterotetrameric glycoproteins of about 150,000 daltons, composed of two identical light (L) chains and two identical heavy (H) chains. In most classes of immunoglobulin molecules, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain (VL) at one end and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J Mol. Biol., 186: 651-663, 1985; Novotny and Haber, Proc. Natl. Acad. Sci. USA, 82: 4592-4596, 1985).

The term “antibody” or “Ab” is used in the broadest sense and specifically covers not only native antibodies but also single monoclonal antibodies (including agonist and antagonist antibodies), antibody compositions with polyepitopic specificity, as well as antibody fragments (e.g., Fab, F(ab′)₂, scFv and Fv), so long as they exhibit the desired biological activity.

“Cytokine” is a generic term for a group of proteins released by one cell population which act on another cell population as intercellular mediators. Examples of such cytokines are lymphokines, monokines, and traditional polypeptide hormones. Included among the cytokines are interferons (IFN, notably IFN-γ), interleukins (IL, notably IL-1, IL-2, IL-4, IL-10, IL-12), colony stimulating factors (CSF), thrombopoietin (TPO), erythropoietin (EPO), leukemia inhibitory factor (LIF), kit-ligand, growth hormones (GH), insulin-like growth factors (IGF), parathyroid hormone, thyroxine, insulin, relaxin, follicle stimulating hormone (FSH), thyroid stimulating hormone (TSH), leutinizing hormone (LH), hematopoietic growth factor, hepatic growth factor, fibroblast growth factors (FGF), prolactin, placental lactogen, tumor necrosis factors (TNF), mullerian-inhibiting substance, mouse gonadotropin-associated peptide, inhibin, activin, vascular endothelial growth factor (VEGF), integrin, nerve growth factors (NGF), platelet growth factor, transforming growth factors (TGF), osteoinductive factors, etc.

The term “subject” as used herein refers to an animal having an immune system, preferably a mammal (e.g., rodent such as mouse). In particular, the term refers to humans.

The term “about” or “approximately” usually means within 20%, more preferably within 10%, and most preferably still within 5% of a given value or range. Alternatively, especially in biological systems (e.g., when measuring an immune response), the term “about” means within about a log (i.e., an order of magnitude) preferably within a factor of two of a given value.

The terms “vector”, “cloning vector”, and “expression vector” mean the vehicle by which a DNA or RNA sequence (e.g., a foreign gene) can be introduced into a host cell, so as to transform the host and promote expression (e.g., transcription and/or translation) of the introduced sequence. Vectors include plasmids, phages, viruses, etc.

In accordance with the present invention there may be employed conventional molecular biology, microbiology, and recombinant DNA techniques within the skill of the art. Such techniques are well-known and are explained fully in the literature. See, e.g., Sambrook, Fritsch and Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

As used herein, the term “polypeptide” refers to an amino acid-based polymer, which can be encoded by a nucleic acid or prepared synthetically. Polypeptides can be proteins, protein fragments, chimeric proteins, etc. Generally, the term “protein” refers to a polypeptide expressed endogenously in a cell. Generally, a DNA sequence encoding a particular protein or enzyme is “transcribed” into a corresponding sequence of mRNA. The mRNA sequence is, in turn, “translated” into the sequence of amino acids which form a protein. An “amino acid sequence” is any chain of two or more amino acids. The term “peptide” is usually used for amino acid-based polymers having fewer than 100 amino acid constituent units, whereas the term “polypeptide” is reserved for polymers having at least 100 such units. Herein, however, “polypeptide” will be the generic term for proteins and peptides as well as polypeptides.

The term “non-replicating” or “replication-impaired” as used herein in relation to viruses and viral vectors means not capable of replication to any significant extent in the majority of normal host cells. Viruses which are non-replicating or replication-impaired may have become so naturally (i.e., they may be isolated as such from nature) or artificially (e.g., by breeding in vitro or by genetic manipulation such as deletion or mutation of a gene which is critical for replication). More precisely, the term “non-replicating” or “replication-impaired” as used herein and as it applies to poxviruses means viruses which satisfy either or both of the following criteria: (i) exhibit an approximately 10-fold reduction in DNA synthesis compared to the Copenhagen strain of vaccinia virus in MRC-5 cells; (ii) exhibit an approximately 100-fold reduction in viral titer compared to the Copenhagen strain of vaccinia virus in HeLa cells. Examples of poxviruses which fall within this definition are MVA, NYVAC and avipox viruses, while a virus which falls outside the definition is the attenuated vaccinia strain M7. There will generally be one or a few cell types in which such non-replicating or replication-impaired viruses can be grown, such as, e.g., chick embryo fibroblast (CEF) cells for MVA.

Antigens

The antigens used in immunogenic (e.g., vaccine) compositions of the instant invention are non-hepatitis B antigens, preferably non-hepadnaviral antigens, which can be derived from a eukaryotic cell (e.g., tumor, parasite, fungus), bacterial cell, viral particle, or any portion thereof.

Examples of preferred non-hepadnaviral antigens of the present invention include (i) protozoan antigens such as those derived from Plasmodium sp., Toxoplasma sp., Pneumocystis carinii, Leishmania sp., and Trypanosoma sp., particularly preferred are malaria-specific antigens, e.g., synthetic peptide antigens comprising at least one CD8+ T cell epitope of the malarial circumsporozoite (CS) protein (see below); (ii) viral protein or peptide antigens such as those derived from influenza virus (e.g., surface glycoproteins hemagluttinin (HA) and neuraminidase (NA) or the nucleoprotein (NP) [e.g., NP CD8+ T cell epitope TYQRTRALV (SEQ ID NO: 44) as described in Bodmer et al., Cell, 52:253, 1988 and Tsuji et al., J. Virol. 72: 6907-6910, 1998 or NP CTL epitopes SDYEGRLI (SEQ ID NO: 45) as described in Gould et al., J. Virol., 65:5401, 1991 and Murata et al., Cell Immunol., 173:96-107, 1996 and ASNENMETM (SEQ ID NO: 46) as disclosed, e.g., in PCT Application No. WO 98/56919]); immunodeficiency virus (e.g., a simian immunodeficiency virus (SIV) antigen [e.g., SIV-env CTL epitope EITPIGLAP (SEQ ID NO: 47) as disclosed, e.g., in PCT Application No. WO 98/56919], or a human immunodeficiency virus antigen (HIV-1) such as gp120 [RGPGRAFVTI (SEQ ID NO: 48) and GRAFVTIGK (SEQ ID NO: 49) CTL epitopes as disclosed, e.g., in PCT Application No. WO 98/56919], gp160, p18 antigen [e.g., CD8+ T cell epitope RGPGRAFVTI (SEQ ID NO: 50)], gp41 [YLKDQQLL (SEQ ID NO: 51) and ERYLKDQQL (SEQ ID NO: 52) CTL epitopes as disclosed, e.g., in PCT Application No. WO 98/56919], Gag p24 CD8+ T cell epitopes [e.g., KAFSPEVIPMF (aa 30-40, SEQ ID NO: 53), KAFSPEVI (aa 30-37, SEQ ID NO: 54), TPQDLNM (or T) ML (aa 180-188, SEQ ID NOS: 55 and 56), DTINEEAAEW (aa 203-212, SEQ ID NO: 57), KRWIILGLNK (aa 263-272, SEQ ID NO: 58), and QATQEVKNW (aa 308-316, SEQ ID NO: 59)], or Gag p17 CD8+ T cell epitopes [e.g., RLRPGGKKK (aa 20-29, SEQ ID NO: 60) and SLYNTVATL (aa 77-85, SEQ ID NO: 61)], Tat, Pol, Nef [e.g., CTL epitopes AVDLSHFLK (SEQ ID NO: 62) and VPLRPMTY (SEQ ID NO: 63) as disclosed, e.g., in PCT Application No. WO 98/56919], and Env [e.g., CTL epitopes ILKEPVHGVY (SEQ ID NO: 64) and VIYQYMDDL (SEQ ID NO: 65) as disclosed, e.g., in PCT Application No. WO 98/56919]; herpesvirus (e.g., a glycoprotein, for instance, from feline herpesvirus, equine herpesvirus, bovine herpesvirus, pseudorabies virus, canine herpesvirus, herpes simplex virus (HSV, e.g., HSV tk, gB, gD), herpes zoster virus, Marek's Disease Virus, herpesvirus of turkeys (HVT), cytomegalovirus (CMV), or Epstein-Barr virus); hepatitis C virus; human papilloma virus (HPV); human T cell leukemia virus (HTLV-1); bovine leukemia virus (e.g., gp51,30 envelope antigen); feline leukemia virus (FeLV) (e.g., FeLV envelope protein, a Newcastle Disease Virus (NDV) antigen, e.g., HN or F); rous associated virus (such as RAV-1 env); infectious bronchitis virus (e.g., matrix and/or preplomer); flavivirus (e.g., a Japanese encephalitis virus (JEV) antigen, a Yellow Fever antigen, or a Dengue virus antigen); Morbillivirus (e.g., a canine distemper virus antigen, a measles antigen, or rinderpest antigen such as HA or F); rabies (e.g., rabies glycoprotein G); parvovirus (e.g., a canine parvovirus antigen); hepatitis C virus (HCV); poxvirus (e.g., an ectromelia antigen, a canary poxvirus antigen, or a fowl poxvirus antigen such as chicken pox virus varicella zoster antigen); infectious bursal disease virus (e.g., VP2, VP3, or VP4); Hantaan virus; mumps virus, and measles virus; (iii) bacterial antigens such as Mycobacterium tuberculosis-specific (e.g., Bacillus Calmette-Guérin [BCG]-38 kD protein; antigen 85 complex [as disclosed in Klein et al., J. Infect. Dis., 183:928-34, 2001], see also a list of antigens in Klein and McAdam, Arch. Inmunol. Ther. Exp. (Warsz.), 47:313-320, 1999), Listeria monocytogenes-specific (e.g., as disclosed in Finelli et al., Immunol. Res., 19:211-223, 1999), Salmonella typhii-specific, Shigella flexineri-specific, staphylococcus-specific, streptococcus-specific, pneumococcus-specific (e.g., PspA [see PCT Publication No. WO 92/14488]), Neisseria gonorrhea-specific, Borrelia-specific (e.g., OspA, OspB, OspC antigens of Borrelia associated with Lyme disease such as Borrelia burgdorferi, n Borrelia afzelli, and Borrelia garinii [see, e.g., U.S. Pat. No. 5,523,089; PCT Application Nos. WO 90/04411, WO 91/09870, WO 93/04175, WO 96/06165, WO93/08306; PCT/US92/08697; Bergstrom et al., Mol. Microbiol., 3: 479-486, 1989; Johnson et al., Infect. and Immun. 60: 1845-1853, 1992; Johnson et al., Vaccine 13: 1086-1094, 1995; The Sixth International Conference on Lyme Borreliosis: Progress on the Development of Lyme Disease Vaccine, Vaccine 13: 133-135, 1995]), A. pertussis-specific, S. parathyphoid A and B-specific, C. diphtheriae-specific, C. tetanus-specific, C. botulinum-specific, C. perifringens-specific, A. anthracis-specific, A. pestis-specific, V. cholera-specific, H. influenzae-specific, T. palladium-specific, Chlamydia trachomatis-specific (e.g., as disclosed in Kim et al., J. Immunol., 162:6855-6866, 1999), and pseudomonas-specific proteins or peptides; (iv) fungal antigens such as those isolated from candida (e.g., 65 kDa mannoprotein [MP65] from Candida albicans), trichophyton, or ptyrosporum, and (v) tumor-specific proteins such as ErbB receptors, Melan A [MART1], gp100, tyrosinase, TRP-1/gp 75, and TRP-2 (in melanoma; for additional examples, see also a list of antigens provided in Storkus and Zarour, Forum (Genova), 2000 July-September, 10(3):256-270); MAGE-1 and MAGE-3 (in bladder, head and neck, and non-small cell carcinoma); HPV EG and E7 proteins (in cervical cancer); Mucin [MUC-1] (in breast, pancreas, colon, and prostate cancers); prostate-specific antigen [PSA] (in prostate cancer); carcinoembryonic antigen [CEA] (in colon, breast, and gastrointestinal cancers), P1A tumor antigen (e.g., CTL epitope LPYLGWLVF [SEQ ID NO: 66] as disclosed in WO 98/56919), and such shared tumor-specific antigens as MAGE-2, MAGE-4, MAGE-6, MAGE-10, MAGE-12, BAGE-1, CAGE-1,2,8, CAGE-3 to 7, LAGE-1, NY-ESO-1/LAGE-2, NA-88, GnTV, and TRP2-INT2 a chimeric tumor CTL epitope string such as MLPYLGWLVF-AQHPNAELL-KHYLFRNL-SPSYVYHQF-IPNPLLGLD (SEQ ID NO: 67) (see, e.g., PCT Application No. WO 98/56919).

The foregoing list of antigens is intended as exemplary, as the antigen of interest can be derived from any animal or human pathogen or tumor. With respect to DNA encoding pathogen-derived antigens of interest, attention is directed to, e.g., U.S. Pat. Nos. 4,722,848; 5,174,993; 5,338,683; 5,494,807; 5,503,834; 5,505,941; 5,514,375; 5,529,780; U.K. Patent No. GB 2 269 820 B; and PCT Publication Nos. WO 92/22641; WO 93/03145; WO 94/16716; WO 96/3941; PCT/US94/06652. With respect to antigens derived from tumor viruses, reference is also made to Molecular Biology of Tumor Viruses, RNA Tumor Viruses, Second Edition, Edited by Weiss et al., Cold Spring Harbor Laboratory Press, 1982. For a list of additional antigens useful in the compositions of the invention see also Stedman's Medical Dictionary (24th edition, 1982).

In a specific embodiment, the compositions of the present invention augment the immunity against malaria in a susceptible mammal, in particular, against the disease induced by the major human plasmodial species, P. falciparum and P. vivax, and murine plasmodial species P. yoelii and P. berghei. These compositions comprise a recombinant hepatitis B core carrier platform and (i) at least one malaria-specific peptide comprising a CD8+ T cell epitope capable of eliciting an anti-malarial T-cell response, preferably in mammals of diverse genetic backgrounds (e.g., YNRNIVNRLLGDALNGKPEEK [SEQ ID NO: 3] or SYVPSAEQI [SEQ ID NO: 1] CD8+ T cell epitopes of P. yoelii CS protein [Renia et al., J. Immunol., 22: 157-160, 1993; Rodrigues et al., Int. Immunol., 3: 579-585, 1991] or SYIPSAEKI [SEQ ID NO: 2] CTL epitope of P. berghei CS protein, or EYLNKIQNSLSTEWSPCSVT [SEQ ID NO: 4] universal T cell epitope of P. falciparum CS protein [Nardin et al., Science 246:1603, 1989; Moreno et al., Int. Immunol. 3: 997, 1991; Moreno et al., J. Immunol. 151: 489, 1993]), or the following CD8⁺ CTL epitopes on P. falciparum preerythrocytic-stage proteins recognized by T cells from volunteers immunized with radiation-attenuated P. falciparum sporozoites (as disclosed in Aidoo et al, Infect. Immun., 68:227-232, 2000 and Kumar et al., Infect. Immun., 69: 2766-2771, 2001):

TABLE 1

| | | |
|---|---|---|
| Sequence | Protein | Residues |

| | | | |
|---|---|---|---|
| KPKDELDYENDIEKKICKMEKCS | (SEQ ID NO: 5) | CS | 368-390 |
| MNHLGNVKYLVIVFL | (SEQ ID NO: 6) | SSP2 | 1-15 |
| EVDLYLLMDCSGSIR | (SEQ ID NO: 7) | SSP2 | 46-60 |
| LLSTNLPYGKTNLTD | (SEQ ID NO: 8) | SSP2 | 121-135 |
| LPYGKTNLTDALLQV | (SEQ ID NO: 9) | SSP2 | 126-140 |
| TNLTDALLQVRKHLN | (SEQ ID NO: 10) | SSP2 | 131-145 |
| ALLQVRKHLNDRINR | (SEQ ID NO: 11) | SSP2 | 136-150 |
| ENVKNVIGPFMKAVC | (SEQ ID NO: 12) | SSP2 | 221-235 |
| CEEERCLPKREPLDV | (SEQ ID NO: 13) | SSP2 | 281-295 |
| CLPKREPLDVPDEPE | (SEQ ID NO: 14) | SSP2 | 286-300 |
| ALLACAGLAYKFVVP | (SEQ ID NO: 15) | SSP2 | 521-535 |
| APFDETLGEEDKIDLD | (SEQ ID NO: 16) | SSP2 | 546-560 |
| TLGEEDKDLDEPEQF | (SEQ ID NO: 17) | SSP2 | 551-565 |
| ASKNKEKAL | (SEQ ID NO: 18) | SSP2 | 107-115 |
| KNKEKALII | (SEQ ID NO: 19) | SSP2 | 109-117 |
| FLIFFDLFLV | (SEQ ID NO: 20) | SSP2 | 14-23 |
| VLAGLLGNV | (SEQ ID NO: 21) | EXP1 | 80-88 |
| GLIMVLSFL | (SEQ ID NO: 22) | CS | 394-402 |
| KILSVFFLA | (SEQ ID NO: 23) | EXP1 | 2-10 |
| GLLGNVSTV | (SEQ ID NO: 24) | EXP1 | 83-91 |
| VLLGGVGLVL | (SEQ ID NO: 25) | EXP1 | 91-100 |
| ILSVSSFLFV | (SEQ ID NO: 26) | CS | |
| QTNFKSLLR | (SEQ ID NO: 27) | LSA1 | 94-102 |
| LACAGLAYK | (SEQ ID NO: 28) | SSP2 | 523-531 |
| VTCGNGIQVR | (SEQ ID NO: 29) | CS | 344-353 |
| ALFFIIFNK | (SEQ ID NO: 30) | EXP1 | 10-18 |
| LLACAGLAYK | (SEQ ID NO: 31) | SSP2 | 522-531 |
| GVSENIFLK | (SEQ ID NO: 32) | LSAI | 105-113 |
| HVLSFINSYEK | (SEQ ID NO: 33) | LSA1 | 59-68 |
| FILVNLLIFH | (SEQ ID NO: 34) | LSA1 | 11-20 |
| MPLETQLAI | (SEQ ID NO: 35) | PfS16 | 77-85 |
| TPYAGEPAPF | (SEQ ID NO: 36) | SSP2 | 539-548 |
| DLLEEGNTL | (SEQ ID NO: 37) | LSA3 | 111-119 |
| KLEELHENV | (SEQ ID NO: 38) | LSA3 | 893-901 |
| VLDKVEETV | (SEQ ID NO: 39) | LSA3 | 981-989 |
| GLLNKLENI | (SEQ ID NO: 40) | LSA3 | 1060-1068 |
| MEKLKELEK | (SEQ ID NO: 41) | LSA3 | 1260-1268 |
| EPKDEIVEV | (SEQ ID NO: 42) | LSA3 | 1524-1532 |
| ATSVLAGL | (SEQ ID NO: 43) | EXP1 | 77-84 |

or additional P. falciparum CTL epitopes disclosed in PCT Application No. WO 98/56919, e.g., KPNDKSLY (SEQ ID NO: 68), KPKDELDY (SEQ ID NO: 69), KPIVQYDNF (SEQ ID NO: 70), ASKNEKALII (SEQ ID NO: 71), GIAGGLALL (SEQ ID NO: 72), MNPNDPNRNV (SEQ ID NO: 73), MINAYLDKL (SEQ ID NO: 74), etc.; and optionally (ii) one or more malaria-specific peptide comprising a non-CD8+ epitope such as, e.g., a T cell epitope (NVDPNANP)ₙ (SEQ ID NO: 75) or a B cell epitope (NANP)ₙ (e.g., (NANP)₃ (SEQ ID NO: 76)) located within the repeat region of the CS protein of P. falciparum (Nardin et al., J. Exp. Med. 156: 20, 1982; Nardin et al., Ann. Rev. Immunol. 11: 687, 1993). B cell epitopes preferably elicit the production of antibodies that specifically recognize and bind to the malarial circumsporozoite (CS) protein. In addition to epitopes derived from the plasmodial circumsporozoite (CS) protein, the compositions of the invention can comprise B cell and T cell epitopes derived from, and reactive with, other malarial components, such as, for example, the Erythrocyte Secreted Protein-1 or -2 (PvESP-1 or PvESP-2) (see, e.g., U.S. Pat. No. 5,874,527), sporozoite surface protein designated Thrombospondin Related Adhesion (Anonymous) protein (TRAP), also called Sporozoite Surface Protein 2 (SSP2), liver stage antigen 1 (LSA-1), liver stage antigen 3 (LSA-3), exported protein 1 (EXP1), hsp70, SALSA, sporozoite threonine- and asparagine-rich protein (STARP), Hep17, MSA, RAP-1, and RAP-2.

The present invention also encompasses B cell and T cell epitopes derived from other plasmodial species, including without limitation P. vivax, P. malariae, P. ovale, P. reichenowi, P. knowlesi, P. cynomolgi, P. brasilianum, and P. chabaudi. These epitopes typically comprise between 8 and 18 amino acid residues, derived from a plasmodial protein.

To provide additional antigen-derived B and T cell epitopes for use in the compositions of the present invention, these epitopes can be identified by one or a combination of several methods well known in the art, such as, for example, by (i) fragmenting the antigen of interest into overlapping peptides using proteolytic enzymes, followed by testing the ability of individual peptides to bind to an antibody elicited by the full-length antigen or to induce T cell or B cell activation (see, e.g., Janis Kuby, Immunology, pp. 79-80, W. H. Freeman, 1992); (ii) preparing synthetic peptides whose sequences are segments or analogs of a given antigen (see, e.g., Alexander et al., Immunity, 1: 751-61, 1994; Hammer et al., J. Exp. Med., 180: 2353-8, 1994), or constructs based on such segments, or analogs linked or fused to a carrier or a heterologous antigen and testing the ability of such synthetic peptides to elicit antigen-specific antibodies or T cell activation (e.g., testing their ability to interact with MHC class II molecules both in vitro and in vivo [see, e.g., O'Sullivan et al., J. Immunol., 147: 2663-9, 1991; Hill et al., J. Immunol., 147: 189-197, 1991]); for determination of T cell epitopes, peptides should be at least 8 to 10 amino acids long to occupy the groove of the MHC class I molecule and at least 13 to 25 amino acids long to occupy the groove of MHC class II molecule, preferably, the peptides should be longer; these peptides should also contain an appropriate anchor motif which will enable them to bind to various class I or class II MHC molecules with high enough affinity and specificity to generate an immune response (see Bocchia et al., Blood, 85: 2680-2684, 1995; Englehard, Ann. Rev. Immunol., 12: 181, 1994); (iii) sequencing peptides associated with purified MHC molecules (see, e.g., Nelson et al., Proc. Natl. Acad. Sci. USA, 94:628-33, 1997); (iv) screening a peptide display library for high-affinity binding to MHC class II molecules, TCR, antibodies raised against a full-length antigen, etc. (see, e.g., Hammer et al., J. Exp. Med., 176:1007-13, 1992); (v) computationally analyzing different protein sequences to identify, e.g., hydrophilic stretches (hydrophilic amino acid residues are often located on the surface of the protein and are therefore accessible to the antibodies) and/or high-affinity TCR or MHC class II allele-specific motifs, e.g., by comparing the sequence of the protein of interest with published structures of peptides associated with the MHC molecules (Mallios, Bioinformatics, 15:432-439, 1999; Milik et al., Nat. Biotechnol., 16: 753-756, 1998; Brusic et al., Nuc. Acids Res, 26: 368-371, 1998; Feller and de la Cruz, Nature, 349: 720-721, 1991); (vi) performing an X-ray crystallographic analysis of the native antigen-antibody complex (Janis Kuby, Immunology, p. 80, W. H. Freeman, 1992), and (vii) generating monoclonal antibodies to various portions of the antigen of interest, and then ascertaining whether those antibodies attenuate in vitro or in vivo growth of the pathogen or tumor from which the antigen was derived (see U.S. Pat. No. 5,019,384 and references cited therein).

In the disclosed compositions, the antigen is present in immunogenically effective amount. For each specific antigen, the immunogenically effective amount is readily determined experimentally (taking into consideration specific characteristics of a given patient and/or type of treatment) using well-known methods. Generally, this amount is in the range of 0.1 μg-100 mg of an antigen per kg of the body weight.

Immunogenic Constructs and Compositions

According to one of the embodiments, to generate a rHEP particle comprising an antigen of interest, the desired epitope sequence is inserted into an HBcAg core sequence to produce a fusion protein. The epitope sequence can be fused to the N-terminus or C-terminus of HBcAg or can be inserted in a HBcAg region between amino acids 75-85 (preferably, between amino acids 78 and 79). According to the present invention, when C-terminal fusions are used, the C-terminus of the HBcAg after amino acid 149 is preferably deleted to allow insertion of the larger heterologous sequences.

In preferred embodiments of the present invention, a single copy of one epitope, several copies of one epitope, or copies of several different epitopes can be inserted into a single region or several regions of the recombinant HBcAg monomer. The amino acids comprising the epitope can be inserted in a manner such that they replace at least some of the amino acids of the HBcAg monomer. The particle of the present invention is thus a highly versatile vehicle for the presentation of epitopes, providing extensive flexibility in the design of immunogenic particles. In preferred embodiments of the present invention, the regions into which the epitopes can be inserted are those which, upon particle assembly, will elicit the strongest CD8+ T cell response to the epitope. Thus, in one of the preferred embodiments, the epitopes of the invention are fused to the C-terminus of the HBcAg monomer. In another preferred embodiment, the epitopes are inserted in the immunodominant loop around HBcAg amino acids 75-85. Epitopes of about 10 to 50 amino acids in length can be efficiently inserted into a recombinant HBcAg monomer. However, epitopes of greater or lesser length can also be inserted. Generally, any length and combination of epitopes can be inserted so long as the monomer that is produced is able to assemble into particles which elicit an immune response.

The methods for construction of DNA plasmid constructs for expression of fusion proteins are well known in the art. Generally, expression control sequences for prokaryotes include promoters, optionally containing operator portions, and ribosome binding sites. Expression vectors compatible with prokaryotic hosts are commonly derived from, for example, pBR322, a plasmid containing operons conferring ampicillin and tetracycline resistance, and the various pUC vectors, which also contain sequences conferring antibiotic resistance markers. These markers can be used to obtain successful transformants by selection. Commonly used prokaryotic control sequences include β-lactamase (penicillinase) and lactose promoter systems, tryptophan (trp) promoter system, lambda-derived A promoter and N gene ribosome binding site, and the hybrid tac promoter derived from sequences of the try and lac UV5 promoters. The preferred promoter for this invention for rHEP transcription is tac.

rHEP when made in, for example, Escherichia coli spontaneously self-assembles into macromolecular core particles. With respect to obtaining particles for use in the practice of the present invention, the means of generating appropriate quantities of particles and purifying them are well known to those of skill in the art. See, for example, U.S. Pat. Nos. 4,356,270 and 4,563,423. In a preferred embodiment of the present invention, the particles of the present invention are produced in a E. coli recombinant system. However, the particles can be also produced by expression of the monomers in a variety of other recombinant expression systems. For example, Salmonella, yeast, insect cells (using for example, a baculovirus expression vector), plant cells (e.g., tobacco, potato, corn, etc.), transgenic animals, or mammalian cell culture systems. Any appropriate expression system that correctly produces the particles of the present invention can be used in the practice of the present invention. Such systems and their use for the production of recombinant proteins are well known to those of skill in the art.

Alternatively, rHEP particles comprising an antigen of interest can be generated by chemically conjugating the desired epitope sequence to the HBcAg core. In this case, the antigen does not have to be a peptide, but can be any chemical entity, such as, for example, a nucleic acid (DNA or RNA), carbohydrate, polysaccharide, glycoprotein, glycolipid, or a combination thereof. Methods of chemical conjugation are well known in the art (see, e.g., U.S. Pat. No. 6,231,863; European Patent No. EP 0421635).

The methods for production of the boosting component according to the present invention are also well known in the art. For example, non-replicating or replication-impaired recombinant poxvirus vectors can be produced by first cloning the antigen sequence into a shuttle vector under the control of a viral promoter and then transfecting the shuttle vector into mammalian cells infected with a viral vector comprising the wild-type nucleotide sequence of the vaccinia strain of choice (e.g., MVA). Upon transfection, viral sequences flanking the promoter, antigen coding sequence, and marker gene of the shuttle vector recombine with the vaccinia vector and produce a recombinant poxvirus which expresses a marker gene (e.g., glucuronidase or β-galactosidase) allowing identification of plaques containing the recombinant virus. Large-scale recombinant poxvirus production and purification methods are also well known in the art (see, e.g., Current Protocols in Protein Sciences, J. Coligan et al. eds., Vol. 1, Ch. 5.10-5.12, 2001, J. Willey and Sons Ltd. Publishers).

As the rHEP particles exert their immunostimulatory activity in combination with a plurality of different antigens, they are therefore useful for both preventive and therapeutic applications. Accordingly, in a further aspect, the invention provides a prophylactic and/or therapeutic method for treating a disease in a mammal comprising administering to said mammal at least one dose of the priming component comprising a recombinant hepatitis B core particle (rHEP) which is a carrier for one or more non-hepatitis B CD8+ T cell epitopes of an antigen. Preferably, administering of the priming component is followed by administering at least one dose of a boosting component comprising a carrier for one or more CD8+ T cell epitopes of the antigen, including at least one CD8+ T cell epitope, which is the same as the CD8+ T cell epitope of the priming component. This method can be useful, e.g., for protecting against and/or treating various infections as well as for treating various neoplastic diseases.

Vaccination and Immunotherapy

Immunogenicity enhancing methods of the invention can be used to combat infections, which include, but are not limited to, parasitic infections (such as those caused by plasmodial species, etc.), viral infections (such as those caused by influenza viruses, leukemia viruses, immunodeficiency viruses such as HIV, papilloma viruses, herpes virus, hepatitis viruses, measles virus, poxviruses, mumps virus, cytomegalovirus [CMV], Epstein-Barr virus, etc.), bacterial infections that involve MHC class I (such as those caused by staphylococcus, streptococcus, pneumococcus, Neisseria gonorrhea, Borrelia, pseudomonas, mycobacteria, Salmonella, etc.), and fungal infections (such as those caused by Candida, Trichophyton, Ptyrosporum, etc.).

Methods of the invention are also useful in treatment of various cancers, which include without limitation fibrosarcoma, myxosarcoma, liposarcoma, chondrosarcoma, osteogenic sarcoma, chordoma, angiosarcoma, endotheliosarcoma, lymphangiosarcoma, lymphangioendothelio-sarcoma, synovioma, mesothelioma, Ewing's tumor, leiomyosarcoma, rhabdomyosarcoma, colon carcinoma, pancreatic cancer, breast cancer, ovarian cancer, prostate cancer, lymphoma, leukemia, squamous cell carcinoma, basal cell carcinoma, adenocarcinoma, hepatocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

In a specific embodiment, the present invention discloses a method for preventing and/or treating malaria in a mammal (e.g., human), wherein said method comprises administering to said mammal (i) a priming component comprising a recombinant hepatitis B core particle (rHEP) which is a carrier for one or more non-hepatitis B CD8+ T cell epitopes of at least one malaria-specific antigen selected from the group consisting of sporozoite antigens in a first amount, and (ii) a boosting component comprising a carrier for one or more CD8+ T cell epitopes of the antigen, including at least one CD8+ T cell epitope which is the same as the CD8+ T cell epitope of the priming component in a second amount; said first and second amounts being effective in combination to enhance the immune response mounted against said antigen by the host. As disclosed in Example 1, infra, the immunization of mice with rHEP particles comprising a CD8+ epitope of the malarial CS protein (followed by boosting with (i) a recombinant vaccinia virus expressing the same epitope or (ii) irradiated sporozoites) leads to an increase in the number of antigen-specific CD8+ T cells and greatly enhances protective anti-malaria immunity.

In another specific embodiment, the present invention discloses a method for preventing and/or treating flu in a mammal (e.g., human), wherein said method comprises administering to said mammal (i) a priming component comprising a recombinant hepatitis B core particle (rHEP) which is a carrier for one or more non-hepatitis B CD8+ T cell epitopes of at least one influenza virus-specific antigen in a first amount, and (ii) a boosting component comprising a carrier for one or more CD8+ T cell epitopes of the antigen, including at least one CD8+ T cell epitope which is the same as the CD8+ T cell epitope of the priming component in a second amount; said first and second amounts being effective in combination to enhance the immune response mounted against said antigen by the host (see also Example 2, infra).

According to the present invention, the priming and boosting components are administered sequentially. To attain the most efficient immune response, the boosting component is preferably administered from two weeks to four months after the priming component.

The methods of the invention can be used in conjunction with other treatments. For example, an anti-cancer treatment using antigen-containing rHEP particles of the present invention can be used in combination with chemotherapy and/or radiotherapy and/or IL-12 treatment. Antiviral vaccines comprising antigen-containing rHEP particles can be used, for example, in combination with IFN-α treatment, or with other antiviral treatments such as acyclovir, idoxuridine, gancliclovir, as well as the existing or emerging nucleoside analogues.

Formulations and Administration

The invention provides pharmaceutical and vaccine formulations containing therapeutics of the invention, which formulations are suitable for administration to elicit an antigen-specific immune response, in particular, CD8+ T cell response, for the treatment and prevention of infectious or neoplastic diseases described above. Compositions of the present invention can be formulated in any conventional manner using one or more physiologically acceptable adjuvants or excipients. Thus, the priming or boosting component of the invention can be formulated for administration by transdermal delivery, or by transmucosal administration, including but not limited to, oral, buccal, intranasal, opthalmic, vaginal, rectal, intracerebral, intradermal, intramuscular, intraperitoneal, intravenous, subcutaneous routes, via scarification (scratching through the top layers of skin, e.g., using a bifurcated needle), by inhalation (pulmonary) or insufflation (either through the mouth or the nose), or by administration to antigen presenting cells ex vivo followed by administration of the cells to the subject, or by any other standard route of immunization.

Preferably, the immunogenic formulations of the invention can be delivered parenterally, i.e., by intravenous (i.v.), subcutaneous (s.c.), intraperitoneal (i.p.), intramuscular (i.m.), subdermal (s.d.), or intradermal (i.d.) administration, by direct injection, via, for example, bolus injection, continuous infusion, or gene gun (e.g., to administer a vector vaccine to a subject, such as naked DNA or RNA). Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water, before use.

The present invention also contemplates various mucosal vaccination strategies. While the mucosa can be targeted by local delivery of a vaccine, various strategies have been employed to deliver immunogenic compositions to the mucosa. For example, in a specific embodiment, the immunogenic polypeptide or vector vaccine can be administered in an admixture with, or as a conjugate or chimeric fusion protein with, cholera toxin, such as cholera toxin B or a cholera toxin A/B chimera (see, e.g., Hajishengallis, J Immunol., 154: 4322-32, 1995; Jobling and Holmes, Infect Immun., 60: 4915-24, 1992; Lebens and Holmgren, Dev Biol Stand 82: 215-27, 1994). In another embodiment, an admixture with heat labile enterotoxin (LT) can be prepared for mucosal vaccination. Other mucosal immunization strategies include encapsulating the immunogen in microcapsules (see, e.g., U.S. Pat. Nos. 5,075,109; 5,820,883, and 5,853,763) and using an immunopotentiating membranous vehicle (see, e.g., PCT Application No. WO 98/0558). Immunogenicity of orally administered immunogens can be enhanced by using red blood cells (rbc) or rbc ghosts (see, e.g., U.S. Pat. No. 5,643,577), or by using blue tongue antigen (see, e.g., U.S. Pat. No. 5,690,938). Systemic administration of a targeted immunogen can also produce mucosal immunization (see, U.S. Pat. No. 5,518,725).

For oral administration, the formulations of the invention can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. The compositions of the invention can be also introduced in microspheres or microcapsules, e.g., fabricated from poly-glycolic acid/lactic acid (PGLA) (see, U.S. Pat. Nos. 5,814,344; 5,100,669 and 4,849,222; PCT Publication Nos. WO 95/11010 and WO 93/07861). Liquid preparations for oral administration can take the form of, for example, solutions, syrups, emulsions or suspensions, or they can be presented as a dry product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound.

For administration by inhalation, the therapeutics according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Compositions of the present invention can also be formulated in rectal compositions such as suppositories or retention enemas, e.g., containing conventional suppository bases such as cocoa butter or other glycerides.

In addition to the formulations described previously, the compositions can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

As disclosed herein, the antigen-containing priming or boosting component of the invention can be also mixed with excipients which are pharmaceutically acceptable and compatible with the active ingredients. Suitable excipients are, for example, water, saline, buffered saline, dextrose, glycerol, ethanol, sterile isotonic aqueous buffer or the like and combinations thereof. In addition, if desired, the preparations can also include minor amounts of auxiliary substances such as wetting or emulsifying agents, pH buffering agents, and/or immune stimulators (e.g., adjuvants) that enhance the effectiveness of the pharmaceutical composition or vaccine. Suitable adjuvants for pharmaceutical and vaccine compositions of the present invention comprise those adjuvants that are capable of enhancing cell mediated responses towards CD8+ T cell epitopes contained in the rHEP particle as well as adjuvants capable of enhancing other T cell responses and the antibody responses against B cell epitopes on the rHEP particle. Adjuvants are well known in the art (Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X). Preferred adjuvants for use with immunogens of the present invention include aluminium or calcium salts (e.g., hydroxide or phosphate salts). Most preferrably, aluminium hydroxide gels such as Alhydrogel can be used. For aluminium hydroxide gels, the rHEP particles are admixed with the adjuvant so that between 50 to 800 μg of aluminium are present per dose, and preferably between 400 and 600 μg. Adjuvants for use with immunogens of the present invention also include water-in-oil emulsions. Preferably, such emulsions comprise squalene and mannide mono-oleate, optionally with squalane, emulsified with the protein in an aqueous phase. Well known examples of such emulsions include Montanide ISA-720, and Montanide ISA-703 (produced by, e.g., Seppic, Castres, France). Most preferably, Montanide ISA-720 is used, and a ratio of oil-to-water of 7:3 (w/w) is used. Other preferred adjuvants of the invention include oil in water emulsions (as disclosed, e.g., in WO 95/17210 and EP 0 399 843) and particulate carriers such as liposomes (as disclosed, e.g., in WO 96/33739). Adjuvants also include, but are not limited to, muramyl dipeptide and saponins such as Quil A, bacterial lipopolysaccharides such as 3D-MPL (3-O-deacylated monophosphoryl lipid A), or TDM. Immunologically active saponin fractions (e.g. Quil A) having adjuvant activity derived from the bark of the South American tree Quillaja Saponaria Molina are particularly preferred. Derivatives of Quil A, for example QS21 (an HPLC purified fraction derivative of Quil A), and the method of its production is disclosed, for example, in U.S. Pat. No. 5,057,540. Amongst QS21 (known as QA21) other fractions such as QA17 are also disclosed. 3 De-O-acylated monophosphoryl lipid A is a well known adjuvant manufactured by Ribi Immunochem, Montana. It can be prepared, e.g., by the methods taught in GB 2122204B. A preferred form of 3 De-O-acylated monophosphoryl lipid A is in the form of an emulsion having a small particle size less than 0.2 μm in diameter (as disclosed, e.g., in EP 0 689 454). QS21 can be particularly useful in the compositions of the present invention as it has been shown to enhance the induction of T cell responses (see, e.g., Stoute et al. New Eng. J. Medicine, 226: 86-91, 1997). However, all other adjuvants can be also used. Other preferred adjuvants include immunostimulatory oligonucleotides (e.g., CpG sequences). Examples of such oligonucleotides are taught, e.g., in WO 98/40100. In preferred methods of using immunostimulatory oligonucleotides, an oligonucleotide is either admixed with the fusion protein, bound to the fusion protein, or bound to a carrier to which the fusion protein is also bound. As a further exemplary alternative, the protein can be encapsulated within microparticles such as liposomes, or in non-particulate suspensions of polyoxyethylene ether (as disclosed, e.g., in UK Patent Application No. GB9807805.8). Particularly preferred adjuvants are combinations of 3D-MPL and QS21 (as disclosed, e.g., in EP 0 671 948), oil in water emulsions comprising 3D-MPL and QS21 (as disclosed, e.g., in WO 95/17210, PCT/EP98/05714), 3D-MPL formulated with other carriers (as disclosed, e.g., in EP 0 689 454), or QS21 formulated in cholesterol containing liposomes (as disclosed, e.g., in WO 96/33739), or immunostimulatory oligonucleotides (as disclosed, e.g., in WO 96/02555). Alternative adjuvants include, e.g., those described in WO 99/52549 as well as immunostimulatory, immunopotentiating, or pro-inflammatory cytokines, lymphokines, or chemokines or nucleic acids encoding them (specific examples include interleukin (IL)-1, IL-2, IL-3, IL-4, IL-12, IL-13, granulocyte-macrophage (GM)-colony stimulating factor (CSF) and other colony stimulating factors, macrophage inflammatory factor, Flt3 ligand, see additional examples of immunostimulatory cytokines in the Section entitled “Definitions”). These additional immunostimulatory molecules can be delivered systemically or locally as proteins or by expression of a vector that codes for expression of the molecule. The techniques described above for delivery of the priming and boosting components of the invention can also be employed for the delivery of additional immunostimulatory molecules.

The invention also provides a pharmaceutical pack or kit comprising one or more containers filled with one or more of the ingredients of the immunogenic formulations of the invention. In a related embodiment, the present invention provides a kit for conferring immunity against a non-hepatitis B antigen in a mammal comprising (i) a pharmaceutical composition comprising a priming component comprising a recombinant hepatitis B core particle (rHEP) which is a carrier for one or more non-hepatitis B CD8+ T cell epitopes of the antigen in a first amount, and (ii) a pharmaceutical composition comprising a boosting component comprising a carrier for one or more CD8+ T cell epitopes of the antigen, including at least one CD8+ T cell epitope which is the same as the CD8+ T cell epitope of the priming component in a second amount; said kit comprising the priming component in a first container, and the boosting component in a second container, and, optionally, instructions for administration of the components; and wherein optionally the containers are in a package. Each container of the kit can also optionally include one or more physiologically acceptable excipients and/or auxiliary substances. Associated with such container(s) can be a notice in the form prescribed by a governmental agency regulating the manufacture, use or sale of pharmaceuticals or biological products, which notice reflects approval by the agency of manufacture, use or sale for human administration.

The compositions can, if desired, be presented in a pack or dispenser device, which can contain one or more unit dosage forms containing the active ingredient (i.e., an antigen). The pack can, for example, comprise metal or plastic foil, such as a blister pack. The pack or dispenser device can be accompanied by instructions for administration. Compositions of the invention formulated in a compatible pharmaceutical excipient can also be prepared, placed in an appropriate container, and labeled for treatment of an indicated condition.

Effective Dose and Safety Evaluations

According to the methods of the present invention, the pharmaceutical and vaccine compositions described herein are administered to a patient at immunogenically effective doses, preferably, with minimal toxicity. As recited in the Section entitled “Definitions”, “immunogenically effective dose” or “therapeutically effective dose” of disclosed formulations refers to that amount of an antigen-containing composition (e.g., priming or boosting component) that is sufficient to produce an effective immune response in the treated subject and therefore sufficient to result in a healthful benefit to said subject.

Following methodologies which are well-established in the art (see, e.g., reports on evaluation of several vaccine formulations in a collaborative effort between the Center for Biological Evaluation and Food and Drug Administration and the National Institute of Allergy and Infectious Diseases [Goldenthal et al., National Cooperative Vaccine Development Working Group. AIDS Res. Hum. Retroviruses, 1993, 9:545-549]), effective doses and toxicity of the compounds and compositions of the instant invention are first determined in preclinical studies using small animal models (e.g., mice) in which these compounds and compositions have been found to be immunogenic and that can be reproducibly immunized by the same route proposed for the human clinical trials.

In a specific embodiment, the efficiency of epitope-specific CD8+ T cell responses to the pharmaceutical and vaccine compositions of the invention is determined by the enzyme-linked immunospot technique (ELISPOT). ELISPOT is a standard method in the art originally developed by the present inventors and their co-workers (Miyahira et al., J. Immunol. Meth., 181: 45-54, 1995) and widely used by others (see, e.g., Guelly et al., Eur. J. Immunol., 32:182-192, 2002; Nikitina and Gabrilovich, Int. J. Cancer, 94:825-833, 2001; Field et al., Immunol. Rev., 182:99-112, 2001; Altfeld et al., J. Immunol., 167:2743-2752, 2001; Skoberne et al., J. Immunol., 167:2209-2218, 2001). This method employs pairs of antibodies, directed against distinct epitopes of a cytokine, and allows the visualization of cytokine secretion by individual T cells following in vitro stimulation with an antigen. ELISPOT has the advantage of detecting only activated/memory T cells and the cytokine release can be detected at the single cell levels, allowing direct determination of T cell frequencies (Czerkinsky et al., J. Immunol. Methods, 25:29, 1988; Taguchi et al., J. Immunol. Methods, 128:65, 1990). The cytokine captured by the immobilized antibody in the ELISPOT assay is detected in situ using an insoluble peroxidase substrate. Thus, the cytokine secretion by individual cells is clearly visualized. The high sensitivity and easy performance, allowing a direct enumeration of peptide-reactive T cells without prior in vitro expansion, make the ELISPOT assay eminently well suited to monitor and measure T cell responses, particularly, CD8+ T cell responses of very low frequencies. According to alternative embodiments, the efficiency of epitope-specific CD8+ T cell responses to the pharmaceutical and vaccine compositions of the invention can be determined using other art-recognized immunodetection methods such as, e.g., ELISA (Tanguay and Killion, Lymphokine Cytokine Res., 13:259, 1994) and intracellular staining (Carter and Swain, Curr. Opin. Immunol, 9:1977, 1997).

As disclosed herein, for any pharmaceutical composition or vaccine used in the methods of the invention, the therapeutically effective dose can be estimated initially from animal models to achieve a circulating plasma concentration range that includes the IC₅₀ (i.e., the concentration of the test compound which achieves a half-maximal inhibition of symptoms). Dose-response curves derived from animal systems are then used to determine testing doses for the initial clinical studies in humans. In safety determinations for each composition, the dose and frequency of immunization should meet or exceed those anticipated for use in the clinical trial.

The dose of antigen(s) and other components in the compositions of the present invention is determined to ensure that the dose administered continuously or intermittently will not exceed a certain amount in consideration of the results in test animals and the individual conditions of a patient. A specific dose naturally varies depending on the dosage procedure, the conditions of a patient or a subject animal such as age, body weight, sex, sensitivity, feed, dosage period, drugs used in combination, seriousness of the disease. The appropriate dose and dosage times under certain conditions can be determined by the test based on the above-described indices and should be decided according to the judgment of the practitioner and each patient's circumstances according to standard clinical techniques. In this connection, the dose of an antigen is generally in the range of 0.1 μg-100 mg per kg of the body weight.

Toxicity and therapeutic efficacy of immunogenic compositions of the invention can be determined by standard pharmaceutical procedures in experimental animals, e.g., by determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred. While therapeutics that exhibit toxic side effects can be used (e.g., when treating severe forms of cancer or life-threatening infections), care should be taken to design a delivery system that targets such immunogenic compositions to the specific site (e.g., lymphoid tissue mediating an immune response, tumor or an organ supporting replication of the infectious agent) in order to minimize potential damage to other tissues and organs and, thereby, reduce side effects. As disclosed herein (see also Background Section and Examples), the rHEP particles of the invention are not only highly immunostimulating at relatively low doses (e.g., 0.1-100 μg per kg of the body weight) but also possess low toxicity and do not produce significant side effects.

As specified above, the data obtained from the animal studies can be used in formulating a range of dosages for use in humans. The therapeutically effective dosage of compositions of the present invention in humans lies preferably within a range of circulating concentrations that include the ED₅₀ with little or no toxicity. The dosage can vary within this range depending upon the dosage form employed and the route of administration utilized. Ideally, a single dose should be used.

### EXAMPLES

The following Examples illustrate the invention without limiting its scope.

### Example 1

Immunization of Mice with rHEP Particles Expressing the CD8+ Epitope of P. yoelii CS Protein Induce CD8+ T Cell-Specific Responses

Methods

Production of rHEP particles. Recombinant hepatitis B core antigen particles (rHEP) containing the SYVPSAEQI (SEQ ID NO: 1) epitope of the P. yoelii circumsporozoite (CS) protein were produced by the following method:

A. Preparation of V7 Cloning Vector. To enable the fusion of T cell epitopes to the carboxy-terminus of a HBc chimera, a new vector, V7, was constructed.

Plasmid vector pKK223-3 (Pharmacia) was modified to form vector pKK223-3N by the establishment of a unique NcoI restriction site to enable insertion of HBc genes as NcoI-HindIII restriction fragments and subsequent expression in E. coli host cells. To modify the pKK223-3 plasmid vector, a new SphI-HindIII fragment was prepared using pKK223-3 as a template and PCR primers pKK223-3/433-452-F 5′-GGTGCATG CAAGGAGATG-3′ (SEQ ID NO: 77) and pKK223-NcoI-mod-R 5′-GCGAAGCTTCG GATCCCATGGTTTTTTCCTCCTTATGTGAAATTGTTATCCGCTC-3′ (SEQ ID NO: 78; the nucleotide changes made to pKK223-3 to form pKK223-3N are underlined). This PCR fragment was cut with the restriction enzymes SphI and HindIII to provide a 467 bp fragment which was then ligated with a 4106 bp fragment of the pKK223-3 vector, to effectively replace the original 480 bp SphI-HindIII fragment. The resultant plasmid (pKK223-3N) has a size of 4573 bp and is therefore 13 bp shorter than the parent plasmid; it contains modified nucleotide sequence upstream of the introduced NcoI site.

Unique EcoRI and SacI restriction sites were inserted between Val-149 and the termination codons and HindIII restriction site to facilitate directional insertion of synthetic dsDNAs into EcoRI-HindIII (or EcoRI-SacI) restriction site. The primers HBc 149/NcoI-F 5′-TTGGGCCATGGACATCGACCCTTA-3′ (SEQ ID NO: 79; restriction site is underlined) and HBc149/SacI-EcoRI-H3-R 5′-CGCAAGCTTAGAGCTCTT GAATTCCAACAACAGTAGTCTCCG-3′ (SEQ ID NO: 80; restriction sites are underlined) were used to amplify the codons encoding amino acids 1-149 of the HBc gene, and simultaneously introduce an NcoI restriction site at the amino-terminus and EcoRI, SacI and HindIII sites at the carboxy-terminus of the 479 bp-long amplification product. The 479 bp fragment was digested with NcoI and HindIII restriction enzymes and cloned into vector pKK223-3N to form vector V7.

To insert T cell epitopes, V7 was digested with EcoRI and HindIII (or EcoRI and SacI) restriction enzymes and synthetic dsDNA fragments having EcoRI/HindIII (or EcoRI/SacI) overhangs, were ligated into V7. For all V7 constructs, the final amino acid of native HBc (Val-149) and the first amino acid of the inserted T cell epitope are separated by a Gly-Ile dipeptide sequence coded for by the nucleotides that form the EcoRI restriction site. For epitopes inserted at EcoRI/SacI, there are additional Glu-Leu residues after the T cell epitope, prior to the termination codons, contributed by the SacI restriction site.

B. Insertion of the CS (252-260) T Cell Epitope into V7. For V7 constructs, synthetic dsDNA fragments coding for the circumsporozoite (CS) protein T cell epitope of interest was inserted into EcoRI/HindIII restriction sites. A synthetic dsDNA fragment encoding the T cell epitope of interest was prepared by mixing complementary single-stranded DNA oligonucleotides at equimolar concentrations, heating to 95° C. for 5 minutes, and then cooling to room temperature at a rate of 1° C. per minute. This annealing reaction was performed in TE buffer. The encoded epitope sequence and the sequences of double-stranded DNAs are shown below (the symbol “#” is used to indicate the presence of a termination codon):

(SEQ ID NO: 81)

I S Y V P S A E Q I #

(SEQ ID NO: 82)

AATTAGCTATGTGCCGTCTGCGGAACAGATTTAATA

(SEQ ID NO: 83)

TCGATACACGGCAGACGCCTTGTCTAAATTATTCGA

Production of rVAC. A recombinant vaccinia virus (rVAC) expressing the SYVPSAEQI (SEQ ID NO: 1) epitope of the P. yoelii circumsporozoite (CS) protein was produced as previously described (see, e.g., Smith et al., Virology, 160:336-345, 1987; Rodrigues et al., J. Immunol., 153:4636-4648, 1994). Briefly, vaccinia virus was derived from the WR strain. An oligonucleotide containing the SalI restriction site, CCACC translational initiator, sequence encoding the SYVPSAEQI epitope, two stop codons (TAGTA), and NotI restriction site was inserted into the multiple cloning site located downstream of the viral early-late promoter P 7.5 in the plasmid pSC11. The resulting plasmid was inserted into the vaccinia thymidine kinase (TK) gene by homologous recombination.

ELISPOT assay for the detection of IFN-γ-producing cells. Essentially, the ELISPOT assay was conducted as previously described (Miyahira et al., J. Immunol. Meth., 181: 45-54, 1995). Ninety-six well nitrocellulose plates (Miliscreen MAHA, Millipore, Bedford UK) were coated with 75 μl of PBS containing 10 μg/ml of anti-mouse interferon-γ (IFN-γ) monoclonal antibody (mAb R4 [EACC]). Following overnight incubation at room temperature, the plates were washed with DMEM-high glucose culture medium containing 5% of fetal calf serum (FCS), and were incubated with DMEM-high glucose culture medium containing 10% FCS, for one hour at 37° C., at least. Duplicates of two-fold dilution series of spleen cells, starting at 1×10⁶ cells/well, were placed in coated plates and co-cultured with 10⁵ irradiated peptide-pulsed P815 target cells at a final 1 μM concentration. Control wells consisted of irradiated P815 target cells without a peptide. The plates were incubated for 24 hours at 37° C. in a 5% CO₂ atmosphere. The plates were washed four times with PBS containing 0.05% of Tween 20 (PBS-TW), and to each one of the wells 100 μl of a biotinylated anti-mouse IFN-γ mAb (XMG1.2 [Pharmingen, CA, USA]) 2.5 μg/ml in PBS-TW was added. Following overnight incubation at 4° C., the plates were further washed four times with PBS-TW. In addition, 100 μl dilution of streptavidin-peroxidase (KPL, Gaithersburg, Md.) at 1:800 was added to each well for one hour at room temperature. The plates were washed four times with PBS-TW and twice with PBS alone, and the spots were developed by adding a solution of Tris 50 mM at pH 7.5, containing 1 mg/ml of the substrate 3-3 diaminobenzidine-tetra-hydrochloride dihydrate and 5 μl of 30% H₂O₂. The number of spots was determined with the aid of a stereomicroscope. For each cell suspension we counted the spots of three different spleen cells dilution stimulated with P815 pulsed with peptide and as control the spleen cells dilution stimulated with P815 not pulsed. As sometimes negative controls display some few spots, a ‘positive’ response is considered that in which the average number of spots plus two standard deviations doubles the numbers of spots observed in negative controls. The number of spots was expressed by the mean amount of IFN-γ secreting cells per 10⁶ spleen cells.

Results

SYVPSAEQI (SEQ ID NO: 1) is the H2K^{d}-restricted epitope located in the P. yoeli circumsporozoite (CS) protein (amino acids 252-260). This epitope is recognized by CD8+ but not by CD4+ T cells (Rodrigues et al., J. Immunol., 153: 4636-4648, 1994). Groups of 3 mice each were immunized with recombinant hepatitis B core antigen particles (rHEP) containing the SYVPSAEQI epitope (50 μg s.c.) or with recombinant vaccinia viruses (rVAC; 3×10⁷ pfu i.p.) expressing the same epitope. Two weeks later, mice that were primed with rHEP were boosted with rVAC and vice versa. One group of mice that received rHEP was also boosted with same immunogen. Ten days after boosting, the frequency of SYVPSAEQI-specific CD8+ T cell responses were determined by ELISPOT assay.

ELISPOT (the enzyme-linked immunospot technique) uses pairs of antibodies, directed against distinct epitopes of a cytokine, and allows the visualization of cytokine secretion by individual T cells following in vitro stimulation with antigen. ELISPOT has the advantage of detecting only activated/memory T cells and the cytokine release can be detected at the single cell levels, allowing direct determination of T cell frequencies (Czerkinsky et al., J. Immunol. Methods, 25: 29, 1988; Taguchi et al., J. Immunol. Methods, 128:65, 1990). Furthermore, this assay has been found to be more sensitive than ELISA (Tanguay and Killion, Lymphokine Cytokine Res., 13: 259, 1994) and intracellular staining (Carter and Swain, Curr. Opin. Immunol, 9:1977, 1997). Importantly, differently to ELISA, the cytokine captured by the immobilized antibody in the ELISPOT assay is detected in situ using an insoluble peroxidase substrate. Thus, the cytokine secretion by individual cells is clearly visualized. The high sensitivity and easy performance, allowing a direct enumeration of peptide-reactive T cells without prior in vitro expansion, make the ELISPOT assay eminently well suited to monitor and measure T cell responses, particularly, CD8+ T cell responses of very low-frequencies.

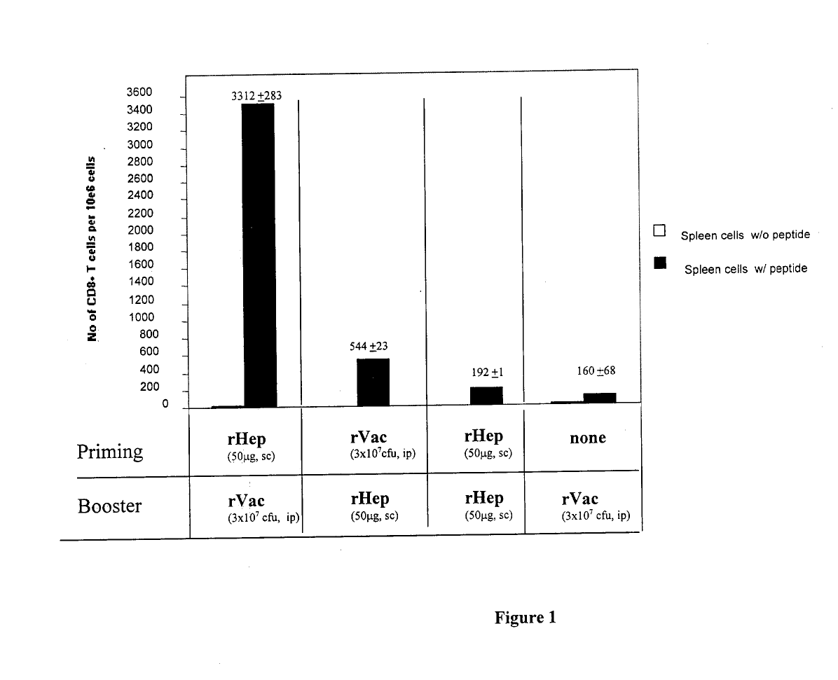

As shown in FIG. 1, priming with rHEP followed by boosting with rVAC induced very high levels of IFN-γ production as determined by ELISPOT assay. This suggests that priming with rHEP induced epitope-specific CD8+ T cells that is recalled after boosting with rVAC. Priming with rVAC and boosting with rHEP as well as double immunization with rHEP induced detectable but much lower levels of epitope-specific CD8+ T cells.

In another experiment, groups of 3 mice each were immunized with irradiated sporozoites (1×10⁵ i.v.) or with rHEP (50 μg s.c.). Three weeks later, mice with that were primed with rHEP were immunized with irradiated sporozoites and vice versa. Eight days after boosting, the frequency of SYVPSAEQI-specific CD8+ T cell responses were determined by ELISPOT assay.

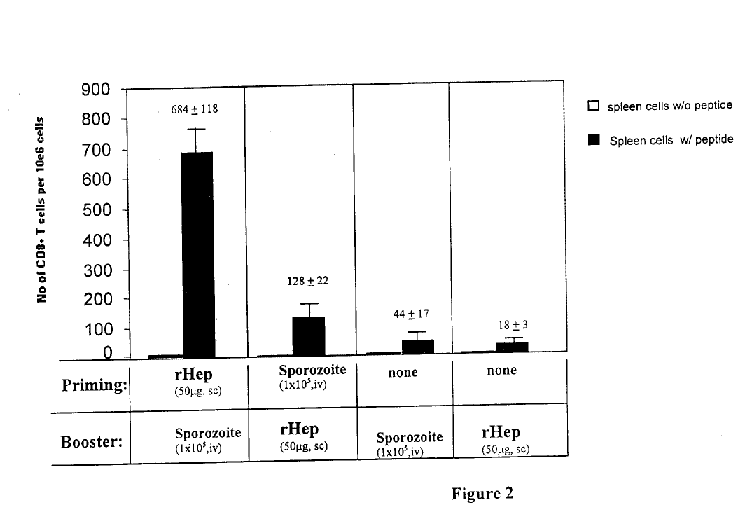

As shown in FIG. 2, rHEP-primed mice immunized with irradiated sporozoites induced high levels of epitope-specific IFN-γ production as determined by ELISPOT assay. This result clearly indicates that CD8+ T cells induced by immunization with synthetic recombinant rHEP particles recognize the sporozoite-derived SYVPSAEQI-epitope after parasite immunization.

Discussion

The results presented above demonstrate that immunization of mice with recombinant hepatitis core particles (rHEP) expressing the SYVPSAEKI epitope of Plasmodium yoelii circumsporozoite protein induce CD8+ T cells specific for the parasites which are detectable after a single immunization.

The particle-induced CD8+ T cell response is boosted after immunization with a recombinant vaccinia virus expressing the same epitope. Most importantly, these particle-induced CD8+ T cells also react and expand in vivo upon immunization with parasite itself thus indicating that these CD8+ T cells, while being induced by a synthetic immunogen, recognize the antigen as expressed in the parasite.

### Example 2

Immunization of Mice with rHEP Particles Expressing the CD8+ Epitopes of Influenza A virus

Methods

Production of CorVax-1690 particles. Recombinant hepatitis B core antigen particles (rHEP-NP) containing the H-2K^{d}-restricted CD8+ T cell epitope IA-NP(147-155) (TYQRTRALV SEQ ID NO: 44; see Bodmer et al., Cell, 52:253, 1988; Tsuji et al., J. Virol. 72: 6907-6910, 1998) of the influenza A virus nucleoprotein (NP) were used to generate recombinant hepatitis core particles (CorVax-1690) following the methods disclosed in Example 1, supra.

Insertion of the IA-NP (147-155) T Cell Epitope into V7 to create CorVax-1690. A synthetic dsDNA fragment encoding the T cell epitope of interest was prepared by mixing complementary single-stranded DNA oligonucleotides at equimolar concentrations, heating to 95° C. for 5 minutes, and then cooling to room temperature at a rate of 1° C. per minute. This annealing reaction was performed in TE buffer. The encoded epitope sequence and the sequences of double-stranded DNAs are shown below (the symbol “#” is used to indicate the presence of a termination codon):

(SEQ ID NO: 84)

I T Y Q R T R A L V #

(SEQ ID NO: 85)

AATTACCTATCAGCGTACGCGCGCGCTGGTGTAGTA

(SEQ ID NO: 86)

TGGATAGTCGCATGCGCGCGCGACCACATCATTCGA

Production of FluVac. A recombinant vaccinia virus expressing the influenza NP protein (FluVac) was produced as previously described (see Example 1, supra, and Smith et al., Virology, 160:336-345, 1987; Rodrigues et al., J. Immunol., 153:4636-4648, 1994). Briefly, the NP gene of Influenza virus strain A/PR/8 was cloned in plasmid pGS69 and inserted into the vaccinia thymidine kinase (TK) gene by homologous recombination using an isolate from WR vaccinia strain as described.

ELISPOT assays for the detection of IFN-γ-producing cells were performed as described in Example 1, supra.

Results

TYQRTRALV (SEQ ID NO: 44) is a H2K^{d}-restricted epitope located in the nucleoprotein (NP) of influenza A virus (amino acids 147-155). Three groups of 3 mice each were immunized with recombinant hepatitis B core antigen particles (CorVax-1690) containing the TYQRTRALV epitope (50 μg s.c.) and one group was immunized with recombinant vaccinia viruses (FluVac; 3×10⁷ pfu i.p.) expressing the entire nucleoprotein (NP) from influenza A virus. Two weeks later, mice that were primed with CorVax-1690 were boosted with either nothing, FluVac, or a recombinant vaccinia virus without an insert (wtVac). Ten days after boosting, the frequency of TYQRTRALV-specific CD8+ T cell responses were determined by ELISPOT assay, as described in Example 1, supra.

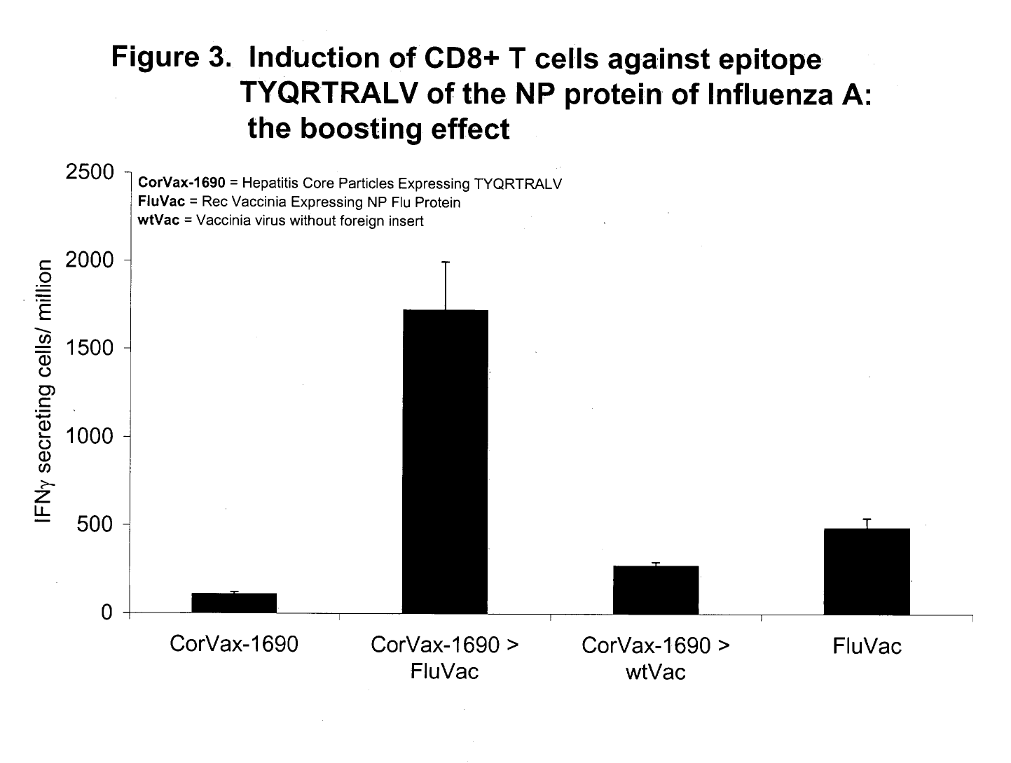

As shown in FIG. 3, priming with CorVax-1690 followed by boosting with FluVac induced very high levels of IFN-γ production as determined by ELISPOT assay compared with mice that were boosted with wtVac. This suggests that priming with CorVax-1690 induced epitope-specific CD8+ T cells that is recalled after boosting with FluVac. Immunization of mice with CorVax-1690 or FluVac alone induced detectable but much lower levels of epitope-specific CD8+ T cells.

Discussion

The results presented above demonstrate that immunization of mice with recombinant hepatitis core particles (CorVax-1690) expressing the TYQRTRALV epitope of influenza A nucleoprotein induce CD8+ T cells. The particle-induced CD8+ T cell response is boosted after immunization with a recombinant vaccinia virus expressing the same epitope in the context of the entire nucleoprotein.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, publications, test methods, literature, and other materials cited herein are hereby incorporated by reference.

What is claimed is:

1. A method for generating an immune response against a non-hepadnaviral antigen in a mammal, which method comprises administering to the mammal at least one dose of a priming component comprising a recombinant hepatitis B core particle (rHEP) which is a carrier for one or more non-hepadnaviral CD8+ T cell epitopes of the antigen, wherein administering the priming component induces an antigen-specific CD8+ T cell immune response.

2. The method of claim 1, wherein the priming component additionally contains at least one non-CD8+ epitope of the antigen.

3. The method of claim 2, wherein the non-CD8+ epitope of the antigen is selected from the group consisting of a CD4+ T cell epitope and a B cell epitope.

4. The method of claim 1, which further comprises administering at least one dose of a boosting component, after the priming component, the boosting component comprising a carrier for one or more CD8+ T cell epitopes of the antigen, including at least one CD8+ T cell epitope which is the same as the CD8+ T cell epitope of the priming component.

5. The method of claim 4, wherein the boosting component additionally contains at least one non-CD8+ epitope of the antigen.

6. The method of claim 5, wherein the non-CD8+ epitope of the antigen is selected from the group consisting of a CD4+ T cell epitope and a B cell epitope.

7. The method of claim 4, wherein the boosting component is a non-replicating or replication-impaired recombinant poxvirus vector.

8. The method of claim 1, wherein the antigen is a protozoan antigen.

9. The method of claim 8, wherein the protozoan antigen is a plasmodial antigen.

10. The method of claim 9, wherein the plasmodial antigen comprises a CD8+ T cell epitope of the plasmodial circumsporozoite (CS) protein.

11. The method of claim 1, wherein the antigen is a viral antigen.

12. The method of claim 11, wherein the viral antigen is an influenza virus-specific antigen.

13. The method of claim 12, wherein the influenza virus-specific antigen comprises a CD8+ T cell epitope of the influenza virus nucleoprotein (NP).

14. A method for treating an infection or cancer in a mammal, which method comprises administering to the mammal at least one dose of a priming component comprising a recombinant hepatitis B core particle (rHEP) which is a carrier for one or more non-hepadnaviral CD8+ T cell epitopes of a non-hepadnaviral antigen, which non-hepadnaviral antigen is an infectious pathogen antigen or a tumor antigen.

15. The method of claim 14, wherein administering of the priming component is followed by administering at least one dose of a boosting component comprising a carrier for one or more CD8+ T cell epitopes of the antigen, including at least one CD8+ T cell epitope which is the same as the CD8+ T cell epitope of the priming component.

16. The method of claim 15, wherein the boosting component is a non-replicating or replication-impaired recombinant poxvirus vector.

17. The method of claim 14, wherein said infection is a parasitic infection.

18. The method of claim 17, wherein said parasitic infection is malaria.

19. The method of claim 14, wherein said infection is a viral infection.

20. The method of claim 19, wherein said viral infection is flu.

21. The method of claim 14, wherein said mammal is human.

22. A pharmaceutical composition comprising an immunogenically effective amount of a priming component comprising a recombinant hepatitis B core particle (rHEP) which is a carrier for one or more non-hepadnaviral CD8+ T cell epitopes of a non-hepadnaviral antigen.

23. The pharmaceutical composition of claim 22 further comprising a pharmaceutically acceptable adjuvant or excipient.

24. The pharmaceutical composition of claim 22, wherein the priming component additionally contains at least one non-CD8+ epitope of the antigen.

25. The composition of claim 24, wherein the non-CD8+ epitope of the antigen is selected from the group consisting of a CD4+ T cell epitope and a B cell epitope.

26. The composition of claim 22, wherein the antigen is a protozoan antigen.

27. The composition of claim 26, wherein the protozoan antigen is a plasmodial antigen.

28. The composition of claim 27, wherein the plasmodial antigen comprises a CD8+ T cell epitope of the plasmodial circumsporozoite (CS) protein.

29. The composition of claim 22, wherein the antigen is a viral antigen.

30. The composition of claim 29, wherein the viral antigen is influenza virus-specific.

31. The composition of claim 30, wherein the influenza virus-specific antigen comprises a CD8+ T cell epitope of the influenza virus nucleoprotein (NP).

32. A method for augmenting immunity induced by an antigen in a mammal comprising administering to said mammal the pharmaceutical composition of claim 22.

33. The method of claim 32 further comprising administering an immunogenically effective amount of a boosting component comprising a carrier for one or more CD8+ T cell epitopes of the antigen, including at least one CD8+ T cell epitope which is the same as the CD8+ T cell epitope of the priming component.

34. The method of claim 33, wherein the boosting component additionally contains at least one non-CD8+ epitope of the antigen.

35. The method of claim 34, wherein the non-CD8+ epitope of the antigen is selected from the group consisting of a CD4+ T cell epitope and a B cell epitope.

36. The method of claim 33, wherein the boosting component is a non-replicating or replication-impaired recombinant poxvirus vector.

37. A method for treating a disease in a mammal comprising administering to said mammal the pharmaceutical composition of claim 22.

38. The method of claim 37, wherein said disease is infection.

39. The method of claim 38, wherein said infection is a parasitic infection.

40. The method of claim 39, wherein said parasitic infection is malaria.

41. The method of claim 38, wherein said infection is a viral infection.

42. The method of claim 41, wherein said viral infection is flu.

43. The method of claim 37, wherein said disease is cancer.

44. The method of claim 37 further comprising administering an immunogenically effective amount of a boosting component comprising a carrier for one or more CD8+ T cell epitopes of the antigen, including at least one CD8+ T cell epitope which is the same as the CD8+ T cell epitope of the priming component.

45. The method of claim 44, wherein the boosting component is a non-replicating or replication-impaired recombinant poxvirus vector.

46. A vaccine composition comprising an immunogenically effective amount of a priming component comprising a recombinant hepatitis B core particle (rHEP) which is a carrier for one or more non-hepadnaviral CD8+ T cell epitopes of a non-hepadnaviral antigen and a pharmaceutically acceptable adjuvant or excipient.

47. The vaccine composition of claim 46, wherein the priming component additionally contains at least one non-CD8+ epitope of the antigen.

48. The vaccine composition of claim 47, wherein the non-CD8+ epitope of the antigen is selected from the group consisting of a CD4+ T cell epitope and a B cell epitope.

49. The vaccine composition of claim 46, wherein the antigen is a protozoan antigen.

50. The vaccine composition of claim 49, wherein the protozoan antigen is a plasmodial antigen.

51. The vaccine composition of claim 50, wherein the plasmodial antigen comprises a CD8+ T cell epitope of the plasmodial circumsporozoite (CS) protein.

52. The vaccine composition of claim 46, wherein the antigen is a viral antigen.

53. The vaccine composition of claim 52, wherein the viral antigen is influenza virus-specific.

54. The vaccine composition of claim 53, wherein the influenza virus-specific antigen comprises a CD8+ T cell epitope of the influenza virus nucleoprotein (NP).

55. A method for conferring immunity against the sporozoite stage of malaria to a susceptible mammalian host comprising administering to said host (i) a priming component comprising a recombinant hepatitis B core particle (rHEP) which is a carrier for one or more non-hepadnaviral CD8+ T cell epitopes of at least one plasmodial sporozoite antigen in a first amount, and (ii) a boosting component comprising a carrier for one or more CD8+ T cell epitopes of the antigen, including at least one CD8+ T cell epitope which is the same as the CD8+ T cell epitope of the priming component in a second amount; said first and second amounts being effective in combination to enhance the immune response mounted against said plasmodial sporozoite antigen by the host.

56. The method of claim 55, wherein the boosting component is a non-replicating or replication-impaired recombinant poxvirus vector.

57. The method of claim 55, wherein said CD8+ T cell epitope has an amino acid sequence selected from the group consisting of SYVPSAEQI (SEQ ID NO: 1), SYIPSAEKI (SEQ ID NO: 2), YNRNIVNRLLGDALNGKPEEK (SEQ ID NO: 3), EYLNKIQNSLSTEWSPCSVT (SEQ ID NO: 4), KPKDELDYENDIEKKICKMEKCS (SEQ ID NO: 5), MNHLGNVKYLVIVFL (SEQ ID NO: 6), EVDLYLLMDCSGSIR (SEQ ID NO: 7), LLSTNLPYGKTNLTD (SEQ ID NO: 8), LPYGKTNLTDALLQV (SEQ ID NO: 9), TNLTDALLQVRKHLN (SEQ ID NO: 10), ALLQVRKHLNDRINR (SEQ ID NO: 11), ENVKNVIGPFMKAVC (SEQ ID NO: 12), CEEERCLPKREPLDV (SEQ ID NO: 13), CLPKREPLDVPDEPE (SEQ ID NO: 14), ALLACAGLAYKFVVP (SEQ ID NO: 15), APFDETLGEEDKDLD (SEQ ID NO: 16), TLGEEDKDLDEPEQF (SEQ ID NO: 17), ASKNKEKAL (SEQ ID NO: 18), KNKEKALII (SEQ ID NO: 19), FLIFFDLFLV (SEQ ID NO: 20), VLAGLLGNV (SEQ ID NO: 21), GLIMVLSFL (SEQ ID NO: 22), KILSVFFLA (SEQ ID NO: 23), GLLGNVSTV (SEQ ID NO: 24), VLLGGVGLVL (SEQ ID NO: 25), ILSVSSFLFV (SEQ ID NO: 26), QTNFKSLLR (SEQ ID NO: 27), LACAGLAYK (SEQ ID NO: 28), VTCGNGIQVR (SEQ ID NO: 29), ALFFIIFNK (SEQ ID NO: 30), LLACAGLAYK (SEQ ID NO: 31), GVSENIFLK (SEQ ID NO: 32), HVLSHNSYEK (SEQ ID NO: 33), FILVNLLIFH (SEQ ID NO: 34), MPLETQLAI (SEQ ID NO: 35), TPYAGEPAPF (SEQ ID NO: 36), DLLEEGNTL (SEQ ID NO: 37), KLEELHENV (SEQ ID NO: 38), VLDKVEETV (SEQ ID NO: 39), GLLNKLENI (SEQ ID NO: 40), MEKLKELEK (SEQ ID NO: 41), EPKDEIVEV (SEQ ID NO: 42), and ATSVLAGL (SEQ ID NO: 43).

58. The method of claim 55, wherein said mammalian host is human.

59. The method of claim 55, wherein said first amount is in the range of 0.1 μg-100 mg per kg of body weight.

60. A kit for conferring immunity against a non-hepadnaviral antigen in a mammal comprising (i) a pharmaceutical composition comprising a priming component comprising a recombinant hepatitis B core particle (rHEP) which is a carrier for one or more non-hepadnaviral CD8+ T cell epitopes of the antigen in a first amount, and (ii) a pharmaceutical composition comprising a boosting component comprising a carrier for one or more CD8+ T cell epitopes of the antigen, including at least one CD8+ T cell epitope which is the same as the CD8+ T cell epitope of the priming component in a second amount; said kit comprising the priming component in a first container, and the boosting component in a second container, and optionally instructions for administration of the components; and wherein optionally the containers are in a package.


SEQUENCE LISTING (86 sequences)

SEQ ID NO: 1 - protein, 9 aa, Plasmodium
SYVPSAEQI

SEQ ID NO: 2 - protein, 9 aa, Plasmodium
SYIPSAEKI

SEQ ID NO: 3 - protein, 21 aa, Plasmodium
YNRNIVNRLLGDALNGKPEEK

SEQ ID NO: 4 - protein, 20 aa, Plasmodium
EYLNKIQNSLSTEWSPCSVT

SEQ ID NO: 5 - protein, 23 aa, Plasmodium
KPKDELDYENDIEKKICKMEKCS

SEQ ID NO: 6 - protein, 15 aa, Plasmodium
MNHLGNVKYLVIVFL

SEQ ID NO: 7 - protein, 15 aa, Plasmodium
EVDLYLLMDCSGSIR

SEQ ID NO: 8 - protein, 15 aa, Plasmodium
LLSTNLPYGKTNLTD

SEQ ID NO: 9 - protein, 15 aa, Plasmodium
LPYGKTNLTDALLQV

SEQ ID NO: 10 - protein, 15 aa, Plasmodium
TNLTDALLQVRKHLN

SEQ ID NO: 11 - protein, 15 aa, Plasmodium
ALLQVRKHLNDRINR

SEQ ID NO: 12 - protein, 15 aa, Plasmodium
ENVKNVIGPFMKAVC

SEQ ID NO: 13 - protein, 15 aa, Plasmodium
CEEERCLPKREPLDV

SEQ ID NO: 14 - protein, 15 aa, Plasmodium
CLPKREPLDVPDEPE

SEQ ID NO: 15 - protein, 15 aa, Plasmodium
ALLACAGLAYKFVVP

SEQ ID NO: 16 - protein, 15 aa, Plasmodium
APFDETLGEEDKDLD

SEQ ID NO: 17 - protein, 15 aa, Plasmodium
TLGEEDKDLDEPEQF

SEQ ID NO: 18 - protein, 9 aa, Plasmodium
ASKNKEKAL

SEQ ID NO: 19 - protein, 9 aa, Plasmodium
KNKEKALII

SEQ ID NO: 20 - protein, 10 aa, Plasmodium
FLIFFDLFLV

SEQ ID NO: 21 - protein, 9 aa, Plasmodium
VLAGLLGNV

SEQ ID NO: 22 - protein, 9 aa, Plasmodium
GLIMVLSFL

SEQ ID NO: 23 - protein, 9 aa, Plasmodium
KILSVFFLA

SEQ ID NO: 24 - protein, 9 aa, Plasmodium
GLLGNVSTV

SEQ ID NO: 25 - protein, 10 aa, Plasmodium
VLLGGVGLVL

SEQ ID NO: 26 - protein, 10 aa, Plasmodium
ILSVSSFLFV

SEQ ID NO: 27 - protein, 9 aa, Plasmodium
QTNFKSLLR

SEQ ID NO: 28 - protein, 9 aa, Plasmodium
LACAGLAYK

SEQ ID NO: 29 - protein, 10 aa, Plasmodium
VTCGNGIQVR

SEQ ID NO: 30 - protein, 9 aa, Plasmodium
ALFFIIFNK

SEQ ID NO: 31 - protein, 10 aa, Plasmodium
LLACAGLAYK

SEQ ID NO: 32 - protein, 9 aa, Plasmodium
GVSENIFLK

SEQ ID NO: 33 - protein, 10 aa, Plasmodium
HVLSHNSYEK

SEQ ID NO: 34 - protein, 10 aa, Plasmodium
FILVNLLIFH

SEQ ID NO: 35 - protein, 9 aa, Plasmodium
MPLETQLAI

SEQ ID NO: 36 - protein, 10 aa, Plasmodium
TPYAGEPAPF

SEQ ID NO: 37 - protein, 9 aa, Plasmodium
DLLEEGNTL

SEQ ID NO: 38 - protein, 9 aa, Plasmodium
KLEELHENV

SEQ ID NO: 39 - protein, 9 aa, Plasmodium
VLDKVEETV

SEQ ID NO: 40 - protein, 9 aa, Plasmodium
GLLNKLENI

SEQ ID NO: 41 - protein, 9 aa, Plasmodium
MEKLKELEK

SEQ ID NO: 42 - protein, 9 aa, Plasmodium
EPKDEIVEV

SEQ ID NO: 43 - protein, 8 aa, Plasmodium
ATSVLAGL

SEQ ID NO: 44 - protein, 9 aa, influenza A virus
TYQRTRALV

SEQ ID NO: 45 - protein, 8 aa, influenza A virus
SDYEGRLI

SEQ ID NO: 46 - protein, 9 aa, influenza A virus
ASNENMETM

SEQ ID NO: 47 - protein, 9 aa, simian immunodeficiency virus
EITPIGLAP

SEQ ID NO: 48 - protein, 10 aa, human immunodeficiency virus (HIV-1)
RGPGRAFVTI

SEQ ID NO: 49 - protein, 9 aa, human immunodeficiency virus (HIV-1)
GRAFVTIGK

SEQ ID NO: 50 - protein, 10 aa, human immunodeficiency virus (HIV-1)
RGPGRAFVTI

SEQ ID NO: 51 - protein, 8 aa, human immunodeficiency virus (HIV-1)
YLKDQQLL

SEQ ID NO: 52 - protein, 9 aa, human immunodeficiency virus (HIV-1)
ERYLKDQQL

SEQ ID NO: 53 - protein, 11 aa, human immunodeficiency virus (HIV-1)
KAFSPEVIPMF

SEQ ID NO: 54 - protein, 8 aa, human immunodeficiency virus (HIV-1)
KAFSPEVI

SEQ ID NO: 55 - protein, 9 aa, human immunodeficiency virus (HIV-1)
TPQDLNMML

SEQ ID NO: 56 - protein, 9 aa, human immunodeficiency virus (HIV-1)
TPQDLNTML

SEQ ID NO: 57 - protein, 10 aa, human immunodeficiency virus (HIV-1)
DTINEEAAEW

SEQ ID NO: 58 - protein, 10 aa, human immunodeficiency virus (HIV-1)
KRWIILGLNK

SEQ ID NO: 59 - protein, 9 aa, human immunodeficiency virus (HIV-1)
QATQEVKNW

SEQ ID NO: 60 - protein, 9 aa, human immunodeficiency virus (HIV-1)
RLRPGGKKK

SEQ ID NO: 61 - protein, 9 aa, human immunodeficiency virus (HIV-1)
SLYNTVATL

SEQ ID NO: 62 - protein, 9 aa, human immunodeficiency virus (HIV-1)
AVDLSHFLK

SEQ ID NO: 63 - protein, 8 aa, human immunodeficiency virus (HIV-1)
VPLRPMTY

SEQ ID NO: 64 - protein, 10 aa, human immunodeficiency virus (HIV-1)
ILKEPVHGVY

SEQ ID NO: 65 - protein, 9 aa, human immunodeficiency virus (HIV-1)
VIYQYMDDL

SEQ ID NO: 66 - protein, 9 aa, Homo sapiens
LPYLGWLVF

SEQ ID NO: 67 - protein, 45 aa, Artificial Sequence
  chimeric tumor CTL epitope string
MLPYLGWLVFAQHPNAELLKHYLFRNLSPSYVYHQFIPNPLLGLD

SEQ ID NO: 68 - protein, 8 aa, Plasmodium
KPNDKSLY

SEQ ID NO: 69 - protein, 8 aa, Plasmodium
KPKDELDY

SEQ ID NO: 70 - protein, 9 aa, Plasmodium
KPIVQYDNF

SEQ ID NO: 71 - protein, 10 aa, Plasmodium
ASKNEKALII

SEQ ID NO: 72 - protein, 9 aa, Plasmodium
GIAGGLALL

SEQ ID NO: 73 - protein, 10 aa, Plasmodium
MNPNDPNRNV

SEQ ID NO: 74 - protein, 9 aa, Plasmodium
MINAYLDKL

SEQ ID NO: 75 - protein, 8 aa, Plasmodium
NVDPNANP

SEQ ID NO: 76 - protein, 12 aa, Plasmodium
NANPNANPNANP

SEQ ID NO: 77 - DNA, 18 bp, Artificial Sequence
  PCR primer pKK223-3/433-452-F
ggtgcatgcaaggagatg

SEQ ID NO: 78 - DNA, 55 bp, Artificial Sequence
  PCR primer pKK223-NcoI-mod-R
gcgaagcttcggatcccatggttttttcctccttatgtgaaattgttatccgctc

SEQ ID NO: 79 - DNA, 24 bp, Artificial Sequence
  PCR primer HBc149/NcoI-F
ttgggccatggacatcgaccctta

SEQ ID NO: 80 - DNA, 42 bp, Artificial Sequence
  PCR primer HBc149/SacI-EcoRI-H3-R
cgcaagcttagagctcttgaattccaacaacagtagtctccg

SEQ ID NO: 81 - protein, 10 aa, Artificial Sequence
  plasmodial CS T cell epitope sequence inserted into V7 constructs
ISYVPSAEQI

SEQ ID NO: 82 - DNA, 36 bp, Artificial Sequence
  strand 1 of dsDNA encoding plasmodial CS T cell epitope sequence for insertion into V7 constructs
aattagctatgtgccgtctgcggaacagatttaata

SEQ ID NO: 83 - DNA, 36 bp, Artificial Sequence
  strand 2 of dsDNA encoding plasmodial CS T cell epitope sequence for insertion into V7 constructs
tcgatacacggcagacgccttgtctaaattattcga

SEQ ID NO: 84 - protein, 10 aa, Artificial Sequence
  sequence encoding IA-NP (147-155) influenza T cell epitope inserted into V7 to create CorVax-1690
ITYQRTRALV

SEQ ID NO: 85 - DNA, 36 bp, Artificial Sequence
  strand 1 of dsDNA encoding IA-NP (147-155) influenza T cell epitope for insertion into V7 to create CorVax-1690
aattacctatcagcgtacgcgcgcgctggtgtagta

SEQ ID NO: 86 - DNA, 36 bp, Artificial Sequence
  strand 2 of dsDNA encoding IA-NP (147-155) influenza T cell epitope for insertion into V7 to create CorVax-1690
tggatagtcgcatgcgcgcgcgaccacatcattcga